(19) United States
(12) Patent Application Publication
(10) Pub. No.: US 2026/0033761 A1
(43) Pub. Date: Feb. 5, 2026

(54) CHARACTERISATION OF NEUROLOGICAL DYSFUNCTION

(71) Applicant: Hoffmann-La Roche Inc., Little Falls, NJ (US)

(72) Inventor: Joerg Felix HIPP, Loerrach (DE)

(21) Appl. No.: 18/998,077

(22) PCT Filed: Jul. 27, 2023
(86) PCT No.: PCT/EP2023/070928

§ 371 Date: Jan. 23, 2025

(30) Foreign Application Priority Data
Jul. 29, 2022 (EP) 22187867.1

(51) Int. Cl. A61B 5/16; A61B 5/00; A61B 5/245; A61B 5/291; A61B 5/374

(52) U.S. Cl. CPC A61B 5/165; A61B 5/245; A61B 5/291; A61B 5/374; A61B 5/7203

(57) ABSTRACT

The invention provides a method of determining whether a subject has a neurological dysfunction associated with a signal in a particular electroencephalogram (EEG) or magnetoencephalogram (MEG) frequency range, the method comprising: obtaining an EEG power spectrum from the subject; and obtaining a metric quantifying the magnitude of power in particular frequency range metric quantifying the power in the power spectrum in the particular frequency range, wherein the metric summarises the power in said frequency range corrected using an estimate of the power in said frequency range that is attributable to background signal that is specific to said frequency range, wherein the metric is indicative of the presence and/or severity and/or direction of a neurological dysfunction. Related methods and devices are also described.

### FIELD OF INVENTION

The present invention relates to a method for determining whether a subject has a particular neurological dysfunction, such as GABA and in particular GABA-A receptor dysfunction, using electroencephalography. It is particularly, but not exclusively, concerned with a method for determining whether a patient with a neuropsychiatric condition or disorder, such as autism spectrum disorder, is likely to be deficient in GABA-A receptor function or to have excessive GABA-A receptor function, and with a method for determining whether a patient is likely to be responsive to a GABA-A modulator. Methods for identifying a treatment or treating a patient accordingly are also described.

### BACKGROUND TO THE INVENTION

Brain activity is characterized by oscillatory electrical activity that can be measured with electroencephalography (EEG). The EEG signals are highly inheritable and therefore characteristic for an individual. In addition, different neuro psychiatric disorders can have EEG signatures that can manifest in frequency-specific changes in EEG power spectra, linked to the pathophysiology. Individual characteristics that are unrelated to e.g. a pathophysiology of interest can therefore lead to considerable variability, impacting the utility of the EEG data to identify a specific pathophysiology.

Additionally, some disorders do not show clear patterns of pathophysiology. For example, autism spectrum disorder (ASD) is a neurodevelopmental condition characterized by core symptoms of persistent deficits in social communication and social interaction, and the presence of restricted, repetitive patterns of behaviours, interests, or activities (American Psychiatric Association, 2013). The prevalence of ASD is estimated to range from 1.68 to 2.24% among school-aged children and to be 1.1% in adults, although data for adult populations are scarce. The diagnosis of ASD is typically made based on behavioural observations. The pathophysiology underlying ASD is poorly understood but likely heterogeneous including different aetiologies. This makes it particularly difficult to treat patients with ASD as not all patients will respond to any particular drug, depending on the drug's mode of action (MoA) and the unknown aetiology of the disease in the particular patient.

Therefore, there is a need for improved methods for identifying groups of patients with a neuropsychiatric disorder with a particular pathophysiology, and for identifying subgroups of patients with neuropsychiatric conditions or disorders including neurodevelopmental disorders such as ASD that may be responsive to drugs with a specific MoA.

### STATEMENTS OF INVENTION

The present inventors postulated that GABAergic dysfunction and in particular GABA-A dysfunction could underlie symptoms in one or more subgroups of ASD. The present inventors further developed a sensitive method for detecting abnormal signal in a selected frequency range of brain oscillatory activity (as recorded e.g. using electroencephalography or magnetoencephalography), such as a particular frequency range of the EEG power spectrum (also referred to as power spectral density), and used this to detect a signal in the beta frequency band indicative of GABAergic dysfunction, with evidence in a plurality of neuropsychiatric disorders including neurodevelopmental disorders that have symptomatic overlap with ASD. This method can therefore be used, amongst other applications, to determine the GABAergic system/GABA-A functional status of a subject, identify subpopulations of subjects with a neuropsychiatric condition, in particular a neurodevelopmental disorder such as ASD, that are likely to have a GABA dysfunction (in particular, a deficient GABA function or an excessive GABA function), to determine whether subjects or populations of subjects are likely to respond to GABA modulators, such as GABA-A modulators and in particular GABA-A a5 modulators, and to inform about the directionality of modulation e.g. agonist/positive allosteric modulators of GABA-A receptors or antagonist/negative allosteric modulators of GABA-A receptors to address reduced and excess GABA-A receptor function, respectively. The present approach also finds uses in analysing MEG (magnetoencephalography) for the purposes mentioned above, as MEG has been shown to also provide signal in particular frequency bands that is indicative of GABA activity (see e.g. Gaetz et al., 2011).

Thus, according to a first aspect, the disclosure provides a method of determining whether a subject has a neurological dysfunction associated with a signal in a particular range of frequency of brain electrophysiological activity, the method comprising: a processor obtaining an EEG and/or MEG power spectrum from the subject; and said processor obtaining a metric quantifying the magnitude of the power in the power spectrum in the particular frequency range, wherein the metric is obtained by summarising the power spectrum in said frequency range corrected using an estimate of the power that is attributable to background signal, wherein the estimate of the power that is attributable to background signal is specific to said frequency range, and wherein the metric is indicative of the presence and/or severity and/or directionality of a neurological dysfunction.

The present inventors have identified that by correcting a signal in an EEG frequency range that is indicative of pathophysiology using a local estimate of the background signal in this range, a more specific indication of the presence of the pathophysiology could be obtained. When applied to a frequency range broadly corresponding to the beta-band range indicative of GABA-A receptor dysfunction, this method was able to reliably identify individuals with GABA-A receptor dysfunction as well as the direction of the disfunction (i.e. deficient or excessive function), to produce a metric that correlates with symptom severity (thereby further strengthening the evidence that the metric is a sensitive and specific indicator of the pathophysiology studied), and identify healthy volunteers that received a GABA-A receptor modulating compound (alogabat). In particular, this metric had superior sensitivity and specificity compared to other spectral analysis metrics even those that use some normalisation, such as e.g. relative power.

The method may have any one or more of the following optional features.

The estimate of the power that is attributable to background signal may comprise a plurality of anchor points wherein anchor points are points of the power spectrum located at or within a predetermined distance of the boundaries of the frequency range. The estimate of the power that is attributable to background signal may comprise a curve in said frequency range of the power spectrum. The curve may be linear, piecewise linear or non-linear. The curve may be derived from the plurality of anchor points or from a global fit of the power spectrum comprising a superposition of oscillatory signals and an aperiodic component.

The estimate of the power that is attributable to background signal may comprise a plurality points obtained from one or more anchor points, wherein anchor points are points of the power spectrum located at or within a predetermined distance of the boundaries of the frequency range, and optionally one or more points that are within said frequency range. The plurality of points may be obtained from said points of the power spectrum by applying respective weights to said points of the power spectrum, wherein the respective weights may be learned or may have been previously learned so as to best separate subjects with a known neurological dysfunction (e.g. subjects with Dup15q, AS, etc.—optionally distinguishing two groups based on the direction of the dysfunction) from typically developing subjects/healthy controls, or to obtain the strongest correlation between the resulting metric and a symptom metric of interest. The term “learn” refers to any machine learning approach suitable to identify optimal values of weights according to an objective criterion such as separation of subjects and/or correlation with symptoms as described above. In such embodiments, determining a summarised estimate of the power in said frequency range that is attributable to background signal that is specific to said frequency range may comprise determining an average of said plurality of points. Similarly, summarising the power in said frequency range may comprise determining an average over a plurality of points, wherein said plurality of points are obtained from a plurality of points of the power spectrum within said frequency range by applying respective weights to said points of the power spectrum, wherein the respective weights may be learned or may have been previously learned so as to best separate subjects with a known neurological dysfunction (e.g. subjects with Dup15q, AS, etc.—optionally distinguishing two groups based on the direction of the dysfunction) from typically developing subjects/healthy controls, or to obtain the strongest correlation between the resulting metric and a symptom metric of interest.

The curve may be linear, piecewise linear or non-linear. For example, a piecewise linear curve may be obtained by determining a power value for each of a plurality of adjacent subranges using a plurality of points in the frequency range and/or within a distance from the boundary of said frequency range (i.e. neighboring frequencies), each subrange comprising at least one point and being associated with a power value determined by weighing the power of the point(s) in the respective subrange using respective weights for each point that are learned so as to best separate subjects with a known neurological dysfunction (e.g. subjects with Dup15q, AS, etc.—optionally distinguishing two groups based on the direction of the dysfunction) from typically developing subjects/healthy controls, or to obtain the strongest correlation between the resulting metric and a symptom metric of interest. Similarly, summarising the power in said frequency range may comprise obtaining a piecewise linear curve and integrating the piecewise linear curve over the frequency range. The piecewise linear curve may be obtained or may have been obtained by determining a power value for each of a plurality of adjacent subranges using a plurality of points in the frequency range, each subrange comprising at least one point and being associated with a power value determined by weighing the power of the point(s) in the respective subrange using respective weights for each point that are learned so as to best separate subjects with a known neurological dysfunction (e.g. subjects with Dup15q, AS, etc.—optionally distinguishing two groups based on the direction of the dysfunction) from typically developing subjects/healthy controls, or to obtain the strongest correlation between the resulting metric and a symptom metric of interest.

Obtaining a metric quantifying the power in the power spectrum in the particular frequency range may comprise: determining a summarised estimate of the power in said frequency range that is attributable to background signal that is specific to said frequency range, summarising the power in said frequency range, and correcting the summarised power in said frequency range using the summarised estimate of the power in said frequency range that is attributable to background signal. Determining a summarised estimate of the power in said frequency range that is attributable to background signal may comprise integrating the estimate of the power in said frequency range that is attributable to background signal over said frequency range and summarising the power in said frequency range comprises integrating the power over said frequency range. Summarising the power in said frequency range may comprise selecting a minimum or maximum of the power in said frequency range or determining an average or weighted average of the power at one or more predetermined frequencies within said frequency range. Determining a summarised estimate of the power in said frequency range that is attributable to background signal may comprise: (i) selecting the estimate of the power that is attributable to background signal at the frequency of the selected minimum or maximum of the power in said frequency range, or (ii) selecting a plurality of anchor points that are on the boundaries or outside of the frequency range and determining an average or weighted average of the signal at said anchor points. Summarising the power in said frequency range may comprise selecting the value of the power at a predetermined frequency in said frequency range, and determining a summarised estimate of the power in said frequency range that is attributable to background signal comprises selecting the estimate of the power that is attributable to background signal at the predetermined frequency. The predetermined frequency may have been previously determined using a plurality of reference power spectra, for example as the frequency at which the power is most likely to have a maximum or minimum value in said plurality of reference power spectra. Summarising the power in said frequency range and determining a summarised estimate of the power in said frequency range that is attributable to background signal may comprise selecting the value of the power and the estimate of the power that is attributable to background signal at a frequency such that the corrected summarised power signal has the minimum or maximum value over the frequency range. Alternatively, obtaining a metric quantifying the power in the power spectrum in the particular frequency range may comprise: determining an estimate of the power in said frequency range that is attributable to background signal that is specific to said frequency range, correcting the power in said frequency range using the estimate of the power in said frequency range that is attributable to background signal in said frequency range, and summarising the corrected power in the frequency range. Summarising the corrected power in the frequency range may comprise selecting the maximum or minimum value of the corrected power in the frequency range, or integrating the corrected power signal over said frequency range. Correcting the power in said frequency range using the estimate of the power in said frequency range that is attributable to background signal in the frequency range may comprise subtracting the estimate of the power attributable to background signal from the power in said frequency range at each frequency in said frequency range or dividing the power signal at each frequency in said frequency range by the estimate of the power attributable to background signal at the corresponding frequency.

Correcting the summarised power in said frequency range using the summarised estimate of power in said frequency range attributable to the background signal may comprise subtracting the summarised estimate of power in said frequency range attributable to background signal in from the summarised metric of the power signal in said frequency range or dividing the summarised power in said frequency range by the summarised estimate of power in said frequency range attributable to background signal. In other words, the metric quantifying the power in the EEG power spectrum in the particular EEG/MEG frequency range may be the maximum or minimum absolute value of the difference (which may be calculated as a ratio, subtraction or any other metric that combines and contrasts two values) between the power signal at each frequency in said frequency range and the estimate of the background signal at the corresponding frequency. Alternatively, the metric quantifying the power in the EEG/MEG power spectrum in the particular frequency range may be the difference between the integral of the power over said frequency range and the integral of the estimate of the power attributable to background signal over said frequency range. Alternatively, the metric quantifying the power in the EEG/MEG power spectrum in the particular frequency range may be the difference between the power and the power attributable to background signal at a pre-defined specific frequency. As another example, the metric quantifying the power in the EEG/MEG power spectrum in the particular frequency range may be the maximum or minimum of the power in the frequency range, corrected using the average or weighted average of the signal at two anchor points (e.g. subtracting the latter from the former or dividing the former by the latter). The two anchor point may be local optima (e.g. minima or maxima) in neighbouring frequency ranges. Neighbouring frequency ranges may be referred to as preceding and subsequent ranges, or ranges around said frequency ranges. They may be immediately adjacent to the said frequency range, but do not need to be. For example, a frequency range may be selected as 16 to 32 Hz, and neighbouring frequency ranges may be selected as 12 to 16 Hz and 32 to 40 Hz, respectively. A weighted average of a plurality of values refers to a weighted sum of said plurality of values divided by the sum of the weights applied in the weighted sum. A weighted average using all equal weights is equivalent to a simple average. For example, a weighted average of a plurality of anchor points may be obtained that weights the plurality of anchor points differently based on the level of confidence in the choice of the anchor points or the likelihood that the chosen anchor points are associated with a signal that is truly attributable to background in said frequency range. For example, a plurality of anchor points may be used and they may be weighted based on the distance between the respective anchor points and the nearest boundary of the frequency range.

The estimate of the power in said frequency range that is attributable background signal may be provided (i.e. may be equal to, derived from, obtained or have been obtained) by interpolation between the power at two or more anchor points that are on the boundaries or outside of the frequency range. The interpolation may be linear interpolation. The interpolation may be in log space, i.e. using log transformed power and frequency values. Interpolation may result in a relationship between frequency and power (i.e. a curve in the power spectrum space) or in a single power value (e.g. using any mathematical relationship that combines the values of the power at the plurality of anchor points). Linear interpolation may result in a linear relationship between frequency and power (i.e. a linear curve in the power spectrum space) or a single power value (e.g. an average or weighted average of the power at a plurality of anchor points). When interpolation results in a single value, this may be used directly as a summarise estimate of the power in said frequency range that is attributable to background signal. The summarised estimate of the power in said frequency range that is attributable to background signal may be determined as a weighted average of the power signal at two or more points that are on the boundaries or outside of the frequency range. One or more of said anchor points may be outside of the frequency range. One or more of said anchor points may be selected in predetermined frequency ranges comprising a preceding frequency range and a subsequent frequency range around the frequency range. One or more of said anchor points may be local optima (minima or maxima) in predetermined frequency ranges comprising a preceding frequency range and a subsequent frequency range around the frequency range. When one or more of said anchor points are outside of the frequency range, they may be within 4 or within 8 Hz of the nearest boundary of the frequency range. When one or more of said anchor points are outside of the frequency range, they may be within a distance to the nearest boundary of the frequency range that is ≤50%, ≤20%, ≤10% of the size of the frequency range. When one or more of said anchor points are outside of the frequency range, they may be within 0.25 octaves or within 0.5 octaves of the nearest boundary of the frequency range. When one or more of said anchor points are outside of the frequency range, they may be within a distance to the nearest boundary of the frequency range that is ≤50%, ≤20%, ≤10% of the size of the frequency range as measured in octaves. When one or more of said anchor points are outside of the frequency range, they may be located such that the range between the plurality of said anchor points includes at most a single complete frequency band and zero, one or two portions of a neighbouring frequency band. The frequency range may be about 16 to about 32 Hz and the anchor points may be about 12 and about 40 Hz. The frequency range may be about 16 to about 32 Hz and the anchor points may be selected in a preceding frequency range that is about 12 to about 16 Hz and/or a subsequent frequency range that is about 32 to about 40 Hz. The predetermined frequency ranges around the frequency range may comprise the frequencies within 2, 4, 5, 6, 7 or 8 Hz of the nearest boundary of the frequency range (individually selected for each of the preceding and subsequent frequency range). The predetermined frequency ranges around the frequency range may each individually extend to include a range of frequencies that is ≤50%, ≤20%, ≤10% of the size of the frequency range. The predetermines frequency ranges may each be selected such that the range between the lower boundary of the preceding frequency range and the upper boundary of the subsequent frequency range includes at most a single complete frequency band and zero, one or two portions of a neighbouring frequency band.

The estimate of the background signal in said frequency range may be provided by (i.e. may be equal to, derived from, obtained or have been obtained) the value of the 1/f^{α} component of a global fit of the power spectrum comprising a superposition of oscillatory signals and a 1/f^{α} component, in said frequency range.

The EEG or MEG power spectrum may be an EEG power spectrum. The power spectrum from the subject may be an average or weighted average power spectrum over a plurality of sensors. Obtaining a power spectrum from the subject may comprise receiving a power spectrum or a plurality of power spectra from the subject from a user interface, memory, database, computing device or EEG/MEG data acquisition means. Obtaining a power spectrum from the subject may comprise receiving a plurality of power spectra comprising a power spectrum for each of a plurality of sensors. The method may further comprise obtaining an average or weighted average of the plurality of power spectra. The EEG or MEG power spectrum may be obtained from EEG/MEG recordings from one or more sensors. The EEG or MEG spectrum may be obtained by combining EEG/MEG spectra from a plurality of sensors, for example by summing said power spectra, averaging said power spectra, or using a weighted average of said power spectra. Alternatively, the methods described herein may be performed separately for a plurality of power spectra each obtained from EEG recordings from one or more sensors, and the resulting metrics may be combined, for example by summing said metrics, averaging said metrics, or using a weighted average of said metrics. A weighted average may weigh some sensors more than others, for example depending on location of sensors that are expected to be more informative. The EEG or MEG power spectrum may be obtained from EEG/MEG recordings from one or more sensors associated with the frontocentral region of the brain. The EEG or MEG power spectrum may be obtained from EEG/MEG recordings from all available sensors, such as e.g. 19 sensors of an EEG 10-20 system. The EEG or MEG power spectrum may be obtained from EEG/MEG recordings from a plurality of sensors comprising sensors associated with the frontocentral region of the brain. The use of frontocentral electrodes may advantageously result in increased signal to noise ratio in some embodiments as these may be less prone to muscle noise, which result in signal in a similar frequency range as the signal associated with some neurological dysfunctions, such as GABA-A dysfunction.

The power spectrum may have been obtained by performing one or more of: obtaining one or more EEG/MEG recordings from the subject, pre-processing the one or more EEG/MEG recordings from the subject, and deriving one or more power spectra from said optionally pre-processed recordings. The power spectrum may be a pre-processed power spectrum. The power spectrum may be a log-scaled power spectrum. The method may comprise applying a non-linear transformation, optionally a log transformation, to the power spectrum prior to obtaining a metric quantifying the power signal in the power spectrum in the particular frequency range. Pre-processing an EEG/MEG recording may comprise one or more of: removing artefactual sections of the recording, wherein artefactual sections refer to signal in the EEG/MEG recording that is not related to brain activity, re-referencing of electrodes, filtering of the EEG/MEG recording, interpolating missing sections, and independent component analysis. Artifactual sections may be excluded or artifacts may be identified by blind source separation or any other method known in the art and then removed from the EEG/MEG recording before deriving a power spectrum.

Said frequency range may be the range between 16 and 32 Hz. Said frequency range may comprise at least a portion of the beta-band. Said frequency range may primarily comprise the beta band. Said frequency range may comprise a portion of the beta-band and optionally one or more portion of one or more neighbouring bands wherein any neighbouring bands represents a minority of the said frequency range. Said frequency range may comprise frequencies in one frequency band. Said frequency range may comprise frequencies in a plurality of frequency bands, at most one of which is completely included in said frequency range. The beta-band may be defined as frequencies between 12 and 30 Hz. The neurological dysfunction may be GABA-A receptor dysfunction. The method may further comprise comparing the value of the metric with a control value or set of control values. The control value or set of control values may correspond to the value(s) of the metric quantified from power spectrum/spectra obtained from healthy individuals, from typically developing individuals, or from subjects with a known neurological dysfunction. The value of the metric and/or the control value or set of values may be adjusted or may have been adjusted based on the age of the subjects or subjects from which the control values were obtained (e.g. by regression) The subjects with a known neurological dysfunction may comprise individuals with a known GABA-A dysfunction, optionally individuals with Dup15q syndrome, Angelman Syndrome, and/or a deletion of the PWAS critical region. The control value may correspond to the summarised estimate of the power in said frequency range that is attributable to background signal. The control value may be the value at which the summarised power in said frequency range is equal to the summarised estimate of the power in said frequency range that is attributable to background signal. The comparison between the value of the metric and the control value or set of control values may be indicative of whether the subject has a neurological dysfunction. The comparison between the value of the metric and the control value or set of control values may be indicative of the extent of the neurological dysfunction. The comparison between the value of the metric and the control value or set of control values may be indicative of the direction of the neurological dysfunction. For example, the value metric for a subject can be compared with the values obtained for a population of healthy or typically developing subjects, and the subject may be determined to have a neurological dysfunction if the value of the metric for the subject is significantly different from the values obtained for a population of healthy or typically developing subjects. As another example, the value metric for a subject can be compared with the values obtained for a population of subjects having a known neurological dysfunction, and the subject may be determined to have a neurological dysfunction if the value of the metric for the subject is not significantly different from the values obtained for a population of healthy or typically developing subjects. As yet another example, the value metric for a subject can be compared with a set of control values comprising one or more values obtained for a population of healthy or typically developing subjects, one or more values obtained for a population of subjects with a known neurological dysfunction, and optionally one or more further sets of values obtained for one or more further populations of subjects with a known neurological dysfunction. The subject may be determined to have a neurological dysfunction or to not have a neurological dysfunction depending on the control values that the value of the metric for the subject is closest to, or the population of control values that it is most statistically likely to belong to. The term “direction” of a neurological dysfunction refers to whether the dysfunction is an excess or deficiency in a particular function. For example, a value of the metric that is higher than a control value or set of control values may be indicative of an excess in the particular neurological function. As another example, a value of the metric that is lower than a control value or set of control values may be indicative of a deficiency in the particular neurological function. The term “extent” of a neurological dysfunction refers to the magnitude or severity of the particular dysfunction. For example, the value of the metric compared to a control value or set of control values may be indicative of the severity of the particular dysfunction, such that larger differences between the value of the metric and the control value(s) are associated with more severe dysfunction than smaller differences between the value of the metric and the control values. The method may further comprise obtaining a power spectrum from the subject, for example by performing and EEG or MEG. Receiving a power spectrum ay comprise receiving the power spectrum from a computing device, user interface, data store or EEG/MEG data acquisition means.

The subject may be a paediatric subject. The subject may be an adult subject. The subject may be a human subject. The subject may be a model animal. The subject may be a mammalian. The subject may be a neuronal cellular culture or brain organoid. The subject may be a subject who has been diagnosed as having or being at risk of having a neuropsychiatric disorder. The subject is a subject who has been diagnosed as having or being at risk of having a neurodevelopmental disorder. The subject may be a subject who has been diagnosed as having or being at risk of having an autism spectrum disorder. The power spectrum may have been recorded over at least 2, 5 or 10 minutes of awake time. The power spectrum may be an EEG power spectrum recorded using at least 19 electrodes mounted according to the 10/20 system. The power spectrum may be an EEG power spectrum recorded using frontocentral electrodes. The power spectrum may have been obtained while the subject is in resting state. The power spectrum may have been obtained while the subject is awake. The filter of the EEG amplifier and/or the sampling rate of the EEG may have been adjusted to capture electrophysiological signals in the frequency range. The subject may be a subject who has been diagnosed as having or being at risk of having a psychiatric disorder (e.g. schizophrenia, bipolar disorder and depression), a neurodegenerative disorder (e.g. Alzheimer's disease, Parkinson's disease, Multiple Sclerosis, amyotrophic lateral sclerosis, Huntington disease) or a neurodevelopmental disorder (e.g. ASD, Dup15q syndrome, Angelman syndrome).

According to a second aspect, there is provided a method of determining whether a subject has a GABA receptor dysfunction, optionally a GABA-A receptor dysfunction. The subject may be a subject who had been diagnosed as having a neuropsychiatric disorder or being likely to have a neuropsychiatric disorder. The neuropsychiatric disorder may be autism spectrum disorder. The method comprises obtaining an EEG and/or MEG power spectrum from the subject; and obtaining a metric quantifying the magnitude of the power in the power spectrum in a particular frequency range, wherein said frequency region comprises at least a portion of the beta band of frequency and/or wherein the beta-band of frequency comprises frequencies between 12 and 32 Hz. The metric may be indicative of the presence and/or severity and/or direction of a GABA receptor dysfunction. The method may have any of the features described in relation to the second aspect. In particular, the metric may be a metric that has the features and/or that has been determined using a method of any embodiment of the first aspect. Thus, also described herein according to the present aspect is a method of determining whether a subject has a GABA receptor dysfunction, the method comprising performing the method of any embodiment of the first aspect, using a power spectrum obtained from said subject, wherein said frequency region comprises at least a portion of the beta band of frequency and/or wherein the beta-band of frequency comprises frequencies between 12 and 32 Hz, wherein the metric is indicative of the presence and/or severity and/or direction of a GABA receptor dysfunction. According to any embodiment of the present aspect, the power spectrum may be an EEG power spectrum. The GABA receptor dysfunction may be a GABA-A receptor dysfunction. Said frequency region may comprise or consist of a range between about 16 Hz and about 32 Hz. A value of the metric that is higher or lower than a control value or set of control values may indicate that the subject has excessive or deficient GABA receptor function, respectively. The magnitude of the difference between the value of the metric and a control value or set of control values may indicate the severity of the dysfunction. The control value may be the value expected for a healthy or typically developing subject. The control value may be a summarised estimate of the power in said frequency range attributable to background signal.

Also described herein according to a third aspect is a method of determining whether a subject with a neuropsychiatric disorder is likely to benefit from treatment with a therapy that treats a neurological dysfunction, the method comprising determining whether the subject has the neurological dysfunction using the method of any embodiment of the first aspect, wherein a subject that has a neurological dysfunction is likely to benefit from treatment with the therapy. As used herein, a neurological dysfunction refers to a particular pathophysiology or brain circuit dysfunction. For example, a neurological dysfunction may be a deficient or excessive function of GABAergic receptors, such as GABA-A receptors.

According to a fourth aspect, there is provided a method of determining whether a subject with a neuropsychiatric disorder is likely to benefit from treatment with a GABA-A activity modulating therapy. The subject may be a subject who has been diagnosed as having an autism spectrum disorder or being likely to have an autism spectrum disorder. The method comprises performing the method of any embodiment of the second aspect using a power spectrum obtained from said subject, and determining whether the subject has a GABA-A receptor dysfunction, wherein a subject that has a GABA-A receptor dysfunction is likely to benefit from treatment with a GABA-A activity modulating therapy. The dysfunction may be an excessive or deficient GABA-A receptor function. The method may comprise predicting the likely magnitude of the benefit of the treatment with a GABA-A activity modulating therapy using the value of the metric quantifying the magnitude of the power in the power spectrum in the particular frequency range. The method may comprise predicting that a subject is not likely to benefit from treatment with a particular type of GABA-A activity modulating therapy, wherein a type of GABA-A activity modulating therapy may be a negative or positive GABA-A activity modulator, wherein a subject that does not have a GABA-A receptor dysfunction is unlikely to benefit from treatment with a GABA-A activity modulating therapy or wherein a subject that has a GABA-A receptor excessive function is unlikely to benefit from treatment with a GABA-A positive modulator or wherein a subject that has a GABA-A receptor defective function is unlikely to benefit from treatment with a GABA-A negative modulator. A subject who is unlikely to benefit from a treatment may be likely to suffer adverse effects of the treatments. The method may comprise predicting the likely magnitude of the adverse effect of the treatment with a GABA-A activity modulating therapy using the value of the metric quantifying the magnitude of the power in the power spectrum in the particular frequency range. A subject with a neuropsychiatric disorder may be a subject who has been diagnosed as having a neuropsychiatric disorder or being at risk of having a neuropsychiatric disorder. The neuropsychiatric disorder may be a psychiatric disorder (e.g. schizophrenia, bipolar disorder and depression), a neurodegenerative disorder (e.g. Alzheimer's disease, Parkinson's disease, Multiple Sclerosis, amyotrophic lateral sclerosis, Huntington disease) or a neurodevelopmental disorder (e.g. ASD, Dup15q syndrome, Angelman syndrome).

The GABA-A activity modulating therapy may be a compound or composition. The GABA-A activity modulating therapy may be a selective GABA-A activity modulating compound or composition. The GABA-A activity modulating therapy may be a GABA-A α5 receptor modulating therapy. The GABA-A activity modulating therapy may be a GABA-A positive modulator or negative modulator. The GABA-A activity modulating therapy may be a GABA-A positive allosteric modulator or negative allosteric modulator. The GABA-A modulating therapy may be applying a positive modulator such as alogabat or a negative modulator such as basmisanil or afizagabar, or a pharmaceutically active salt of any thereof. The method may be performed prior to administering a GABA-A modulating therapy. The method may be performed after administering a GABA-A modulating therapy. The method may be performed after administering an acute GABA-A modulating therapy. The method may be performed after administering a course of a GABA-A modulating therapy. The method may further comprise recommending a GABA-A activity modulating therapy or selecting the subject for treatment with a GABA-A modulating therapy or treating the subject with a GABA-A modulating therapy. A subject that is determined to have excessive GABA-A receptor function may be recommended or selected for treatment or treated with a GABA-A negative modulator. A subject that is determined to have deficient GABA-A receptor function may be recommended or selected for treatment or treated with a GABA-A positive modulator. A subject that is determined to not have deficient GABA-A receptor function may be recommended or selected for treatment or treated with a therapy that is not a GABA-A modulating therapy. The method may be performed at least at two time points comprising a time point prior to administering a GABA-A modulating therapy and/or at one or more time points subsequent to administering a GABA-A modulating therapy. The method may comprise recommending that the GABA-A modulating therapy is discontinued or discontinuing a GABA-A modulating therapy if the comparison between the metric obtained at two time points does not indicate a reduction of GABA-A dysfunction after administering the GABA-A modulating therapy. The method may be performed at least at two time points comprising a time point prior to administering a GABA-A modulating therapy and/or at one or more time points subsequent to administering a GABA-A modulating therapy. The method may comprise recommending that a GABA-A modulating therapy, such as but not limited to the GABA-A modulating therapy, is continued or continuing a GABA-A modulating therapy if the comparison between the metric obtained at two time points does not indicate a reduction of GABA-A dysfunction after administering the GABA-A modulating therapy. The method may be performed using a power spectrum acquired after acute administration of a particular GABA-A activity modulating therapy. The method may further comprise recommending a GABA-A activity modulating therapy or the particular GABA-A activity modulating therapy, recommending that the subject not be treated with a GABA-A activity modulating therapy or the particular GABA-A activity modulating therapy, selecting the subject for treatment without a GABA-A modulating therapy or the particular GABA-A activity modulating therapy, treating the subject with a course of treatment that does not comprise a GABA-A modulating therapy or the particular GABA-A activity modulating therapy. A subject that is determined to have GABA-A receptor dysfunction after acute administration of the GABA-A activity modulating therapy may be recommended or selected for treatment or treated with a course of treatment that does not include a GABA-A activity modulating therapy. A subject that is determined not to have deficient GABA-A receptor function after acute administration of the GABA-A activity modulating therapy may be recommended or selected for treatment or treated with a GABA-A activity modulating therapy.

Thus, the disclosure provides methods to identify subpopulations of subjects with excessive GABA-A function compared to control, methods to identify subpopulations of patients with deficient/insufficient GABA-A function compared to control, methods to determine whether a subject has excessive GABA-A function compared to control, methods to determine whether a subject has deficient/insufficient GABA-A function compared to control, methods to determine whether a subject has a GABA-A function similar to subjects that have excessive GABA-A function (e.g. compared to healthy or typically developing subjects), and methods to determine whether a subject has a GABA-A function similar to subjects that have deficient GABA-A function (e.g. compared to healthy or typically developing subjects).

Also described herein according to a fifth aspect is a method for selecting a subject for participating in a clinical trial for a therapy that treats a neurological dysfunction, the method comprising performing the method of any embodiment of the first or third aspects, and selecting a subject for participating in the clinical trial if the subject is determined to have the neurological dysfunction or to be likely to respond to a therapy that treats the neurological dysfunction.

According to a sixth aspect, there is provided GABA-A modulating therapy for use in a method of treatment of a neuropsychiatric disorder in a subject, the method comprising: (i) determining whether the subject has a GABA-A dysfunction or is likely to benefit from treatment with a GABA-A activity modulating therapy using the method of any of embodiment of the fourth aspect; and (ii) administering the GABA-A modulating therapy to said subject if the subject is determined to have a GABA-A dysfunction or to be likely to benefit from treatment with a GABA-A activity modulating therapy. The GABA-A modulating therapy may be a positive modulator such as alogabat or a negative modulator such as basmisanil.

According to a further aspect, there is provided a method of selecting a subject having an autism spectrum disorder for treatment with a GABA-A modulating therapy, the method comprising determining whether the subject has a GABA-A receptor dysfunction using a method of any embodiment of the second aspect, and selecting the subject for treatment with the GABA-A modulating therapy if the subject is determined to have a GABA-A receptor dysfunction.

According to a further aspect, there is provided a GABA-A modulating therapy for use in a method of treatment of ASD in a subject from whom an EEG power spectrum has been obtained and the subject has been characterised by a method according to the second aspect as having a GABA-A receptor dysfunction.

According to a further aspect, there is provided a method of treating ASD in a subject determined to have a GABA-A receptor dysfunction, wherein the subject is characterised as having a GABA-A receptor dysfunction using the method of any embodiment of the second aspect, and wherein the method comprises treating the subject with a GABA-A modulating therapy.

According to any embodiment of any aspect, the method may further comprise providing to a user, through a user interface, one or more of the value of the metric quantifying the power signal in the EEG power spectrum in the particular EEG spectral frequency range, an indication of whether the subject has a neurological dysfunction, an indication of the neurological dysfunction that a subject is determined to have, an indication of whether the subject is likely to respond to a therapy that treats a neurological dysfunction, a treatment recommendation, an indication of whether a subject should be included for participation in a clinical trial, and a report comprising any one or more of the above.

According to a further aspect, there is provided a system comprising: a processor; and a computer readable medium comprising instructions that, when executed by the processor, cause the processor to perform the (computer-implemented) steps of the method of any preceding aspect. The system may further comprise an EEG/MEG data acquisition means, such as an electroencephalograph. According to a further aspect, there is provided a non-transitory computer readable medium or media comprising instructions that, when executed by at least one processor, cause the at least one processor to perform the method of any embodiment of any aspect described herein. According to a further aspect, there is provided a computer program comprising code which, when the code is executed on a computer, causes the computer to perform the method of any embodiment of any aspect described herein.

### BRIEF DESCRIPTION OF THE FIGURES

FIG. 1 is a flow diagram showing, in schematic form, a method of determining whether a subject has a neurological dysfunction, such as a GABAergic dysfunction or a GABA-A receptor dysfunction, according to the disclosure.

FIG. 2 is a flow diagram showing, in schematic form, a method of characterising a subject as likely or unlikely to respond to a therapy, and/or selecting a subject for participating in a clinal trial, and/or monitoring a subject, according to the disclosure.

FIG. 3 shows an embodiment of a system for characterising the neurological function status of a subject.

FIG. 4 shows the two variants of a method for isolating oscillatory activity from the background signal as described herein (“locally referenced power”) applied to EEGs from individuals with Dup15q syndrome and typical developing controls (TD). In the example shown, all power spectra are averaged across electrodes from a centro-frontal region of interest depicted as an inlet in B. (A) Raw power spectra from individuals with Dup15q syndrome (thin lines) and typical developing controls (bold dashed lines). (B) Variant 1: Illustration of derivation of “locally referenced beta power” using an example power spectrum from an 8 year old individual with Dup15q syndrome. In this variant, the estimate of the background power spectral density (black dotted line between 12 and 40 Hz) is derived via linear interpolation of logarithmic power and frequency with the “support points” at 12 and 40 Hz. The estimated background beta power is shown as a black surface (integral over beta frequency range, i.e. 16-32 Hz). The “locally referenced beta power” is depicted as a hatched surface (absolute beta-band power would be the sum of the black and the hatched surface). The background power spectral value at the peak beta frequency (i.e. frequency where the power spectral density is maximal in the frequency range 16-32 Hz) and the corresponding value for the background are shown as black dots and could be used to derive “locally referenced beta peak power” accordingly. (C). Variant 2: In this variant, the estimate of the background power spectral density (black dotted line) is using the 1/f^{α} component of a global fit of the power spectrum comprising a superposition of oscillatory signals and a 1/f^{α} component (as described in Donoghue et al., 2020). “locally referenced beta power” and “locally referenced beta peak power” can then be derived like in C. (D). Illustration of an intermediate step of deriving “locally referenced beta peak power” using the method variant described in B. From the individual power spectra shown in A, the interpolated (and extrapolated) estimates of the background activity are subtracted. This intermediate steps illustrate how the difference between Dup15q syndrome and TDs becomes more pronounced in the beta frequency range. (E) Same as in D but zoom into the frequency range of most interest.

FIG. 5 shows results of extraction of “locally referenced beta band power” using the variant shown in FIG. 4B for six individuals with Dup15q syndrome and two typical developing controls (on grey backdrop).

FIG. 6 shows (A) “Locally referenced beta power” derived as described in FIG. 4B for individuals with Dup15q syndrome (black shapes) and typical developing controls (hatched shapes) as a function of age. Longitudinal data from the same individuals are connected by lines. Data from individuals with and without a history of epilepsy are depicted by stars and circles, respectively. (B) “Locally referenced beta power” for individuals with Dup15q syndrome, TD controls and subgroups with different genotypes. Effect sizes (ES) between selected groups are shown in the graph (see Table 2 for ES of additional contrasts). (C) Topography of effect size for differences Dup15q vs typical developing controls and for IDIC vs INT. (D,E) Like in B for two variants of “Locally referenced beta power” (D: “Locally referenced beta peak power” as described in 4B; E “Locally referenced beta power” derived from global 1/f fit as described in 4D) and for traditional measures (F: Absolute beta power; G: Relative beta power, i.e. beta power divided by overall signal power). Abbreviations: INT: Interstitial duplications; IDIC: Isodicentric duplications. TD: typical developing. ES: effect size. D-E show the robustness of the findings to the particular variant of the methods described herein.

FIG. 7 shows the correlation of “Locally referenced beta power” (variant described in FIG. 4B) with different clinical scales assessing symptom severity: FSDQ/NVDQ/VDQ, i.e., full-scale, non-verbal and verbal developmental quotient (ratio of developmental age and the chronological age times 100, derived from either the MSEL, i.e., “Mullen scales of early learning” or the DAS-II, i.e., differential ability scales) and VABS adaptive behaviour composite (ABC) and communication, daily living skills and socialization standard sores (i.e., the vineland adaptive behaviour scales; standard scores either derived from the VABS II or VABS 3). The analysis includes individuals with Dup15q syndrome with interstitial (black circles) and isodicentric (white circles) duplications without epilepsy. Both the “Locally referenced beta power” and the clinical scores have been corrected for effects of age (log-age regression) and z-scored separately for interstitial and isodicentric duplications. Z-scores were re-scaled to the mean values and standard deviations for illustration. These steps ensure that correlations are not driven by maturational trajectories or differences between isodicentric and interstitial groups. Data points from the same individuals are connected by lines. Sensitivity analyses show that correlations are significant (all p<0.01) and are of similar magnitude if age correction is not performed or performed jointly for both genotypes. Correlation coefficients are much smaller and not significant for absolute beta power (r<−0.33; p>0.05) except for NVDQ (r=−0.35; p=0.047).

FIG. 8 shows the results of the method for isolating oscillatory activity from the background signal as described herein (“locally referenced power”) applied to EEGs from individuals with Angelman syndrome with deletion genotype and typical developing controls (complement to FIG. 4 where data for Dup15q syndrome and typical developing controls are shown). All power spectra are averaged across electrodes from a centro-frontal region of interest depicted as an inlet in B. (A) Raw power spectra from individuals with Angelman syndrome (thin lines) and typical developing controls (bold dashed lines). (B) Illustration of derivation of “locally referenced beta power” using an example power spectrum from a 14 year old individual with Angelman syndrome (details in FIG. 4B). (C,D) Illustration of an intermediate step of deriving “locally referenced beta peak power”. From the individual power spectra shown in A (details in FIGS. 4D,E).

FIG. 9 shows extraction of “locally referenced beta band power” as described in FIG. 8B for six individuals with Angelman syndrome (deletion genotype) and two typical developing controls (on grey backdrop).

FIG. 10 shows “Locally referenced beta power” derived as described in FIG. 8B for individuals with Angelman syndrome of deletion genotype (black shapes) and typical developing controls (hatched shapes) as a function of age. Data from individuals with and without a history of epilepsy are depicted by stars and circles, respectively. (B) “Locally referenced beta power” for individuals with Angelman syndrome and TD controls. Effect sizes (ES) between groups are shown in the graph. (C) Topography of effect size for differences Angelman syndrome vs typical developing controls. (D) “Locally referenced beta peak power” derived from global 1/f fit as described in 4D. (E) Absolute beta power. (F) Relative beta power, i.e. beta power divided by overall signal power.

FIG. 11 shows the pharmacodynamics EEG effects of the GABA-A a5 receptor positive allosteric modulator (PAM) alogabat recorded in the Phase 1 SAD/MAD study BP40091 in healthy volunteers. (A) Change from baseline in beta-band power (12-30 Hz) across different dose levels and placebo. 25 to 100 mg is a dose range that exhibits the full PD response and is within the GABA-A a5 receptor selective dose range. (B) Change in the power spectrum in response to the GABA-A a5 receptor selective doses (25-100 mg) and placebo relative to pre-treatment baseline. A strong and consistent increase in beta-band power is apparent in the treated group but not in the placebo group. (C-F) Absolute beta power (C), relative beta power (D), “Locally referenced beta power” using the variant described in FIG. 4B (E) and “Locally referenced beta power” using the variant described in FIG. 4C for the placebo and treated group (selective dose range). Effect sizes (ES) are used to quantify the separation between placebo and treated group, while no pre-treatment baseline was considered. Absolute beta power does not allow separating groups, while both “Locally referenced beta power” provide significant separation (ES: ˜0.7, p<0.007). Relative power also allows separation (ES: 0.62, p=0.01) but is not a measure specific to the beta-band, changes at other frequencies may contribute to this effect.

FIG. 12 summarises the results of FIGS. 4 to 6 and shows that a method for isolating oscillatory activity as described herein (“locally referenced power”) clearly separates subjects with neurodevelopmental disorders (Angleman Syndrome and Dup15q Syndrome) from typically developing subjects (top panel). By contrast, a prior art method for quantifying beta-band power does not result in such a clear separation (bottom panel).

FIG. 13 illustrates schematically how the insights described herein (presence of an EEG beta-Band Signature) can be used to identify subgroups in idiopathic ASD that may benefit from enhancing or reducing GABAergic signalling. In particular, individuals with autism spectrum disorder (ASD) may be characterised using a method as described herein to identify subgroups of patients that are “Dup15q-like” (patients with ASD with a beta-band signature similar to patients with Dup15q), i.e. have excess GABA-A receptor function and may benefit treatments reducing GABA function and/or patients that are “Deletion AS-like” (patients with ASD with a beta-band signature similar to patients with deletion AS), i.e. have reduced GABA-A receptor function and may benefit from treatments enhancing GABA-A receptor function. In particular, the former may benefit from treatment with a GABA_{A} a5 negative allosteric modulator (NAM), while the latter may benefit from treatment with a GABA_{A} a5 positive allosteric modulator (PAM).

FIG. 14 shows the hypothetical outcome of a clinical trial with a baseline biomarker, e.g. “locally referenced beta band power” derived from a resting state EEG recording at pre-drug baseline, on the x-axis and the treatment outcome on the y-axis (e.g. change in VABS adaptive behavior composite or grows scores of individuals VABS domains assessed at the end of treatment and before treatment). The response in the placebo group (empty circles) is independent of the biomarker, while there is a relationship between clinical outcome and biomarker values for the treatment group (filled circles; positive slope solid line). Using data collected in such an experiment/clinical trial, a BM threshold can be defined. Individuals with BM values above the threshold are likely to benefit. Instead of a threshold some sort of probability or likelihood value can be derived from the biomarker that indicates treatment outcome. Such a biomarker could then be developed as a complementary or companion diagnostic. The biomarker may also identify individuals with a detrimental response to the drug, i.e. falling below the placebo group. In this case the biomarker could be used to exclude participants.

### DETAILED DESCRIPTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein “treatment” refers to reducing, alleviating or eliminating one or more symptoms of the disease which is being treated, relative to the symptoms prior to treatment. The term “therapy” as used herein typically refers to a compound or composition (also referred to herein as a “drug”) that has a particular function. For example, a GABA activity modulating therapy refers to a compound or composition that is able to modulate the activity of a GABAergic receptor in a subject, when administered to the subject in an appropriate dose (e.g. a therapeutically effective dose). Similarly, a GABA-A activity modulating therapy refers to a compound or composition that is able to modulate the activity of the GABA-A receptor in a subject, when administered to the subject in an appropriate dose (e.g. a therapeutically effective dose). A GABA activity modulating therapy may have specificity for one or more types of GABA receptors (e.g. GABA-A receptors) or subtypes of GABA receptors (e.g. GABA-A a5 receptors). A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

A neuropsychiatric condition or disorder as used herein refers to a disorder that effects brain function, including neurodevelopmental disorders, psychiatric disorders and neurodegenerative disorders. Neuropsychiatric disorders include disorders or conditions that may be referred to as neurological, psychiatric (e.g. schizophrenia, bipolar disorder and depression), neurodegenerative (e.g. Alzheimers disease, Parkinson's disease, Multiple Sclerosis, amyotrophic lateral sclerosis, Huntington disease) and neurodevelopmental (e.g. ASD, Dup15q syndrome, Angelman syndrome) disorders. A neurodevelopmental condition or disorder (NDD) as used herein refers to a condition or disorder that affects brain function, with onset in the developmental period (e.g. from childhood to adolescence and young adulthood). NDDs comprise disorders such as Autism Spectrum Disorder (ASD), Attention-Deficit/Hyperactivity Disorder (ADHD), Neurodevelopmental Motor Disorders, including Tic Disorders (e.g. Tourette Syndrome), intellectual disability, and Schizophrenia. Some NDDs are rare inheritable disorders (i.e. disorders with a known genetic link), such as Fragile X syndrome, Dup15q syndrome, Angelman syndrome.

The systems and methods described herein may be implemented in a computer system, in addition to the structural components and user interactions described. As used herein, the term “computer system” includes the hardware, software and data storage devices for embodying a system or carrying out a method according to the above described embodiments. For example, a computer system may comprise one or more central processing units (CPU) and/or graphics processing units (GPU), input means, output means and data storage, which may be embodied as one or more connected computing devices. Preferably the computer system has a display or comprises a computing device that has a display to provide a visual output display. The data storage may comprise RAM, disk drives, solid-state disks or other computer readable media. The computer system may include a plurality of computing devices connected by a network and able to communicate with each other over that network. It is explicitly envisaged that computer system may consist of or comprise a cloud computer.

The methods described herein are computer implemented unless context indicates otherwise. Indeed, the amount of data involved in analysis of brain oscillatory activity is such that the methods described herein are far beyond the capability of the human brain and cannot be performed as a mental act. The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

The disclosure relates to analysis of brain oscillatory activity (as recorded e.g. using technologies that measure brain electrophysiological signals, such as electroencephalography or magnetoencephalography). For example, methods of analysis of electroencephalogram (EEG) or magnetoencephalography (MEG) data are described, which quantify the intensity of an oscillatory signal in a particular range of frequencies. Such a quantification can be performed by analysis of power spectrum data obtained by electroencephalography or MEG. Electroencephalography is a method to record electrical activity in the brain by measuring voltage fluctuations resulting from ionic currents within the neurons of the brain, using electrodes placed on the scalp. An electroencephalogram comprises data for each of one or more electrodes, quantifying the voltage fluctuation (typically in pV) as a function of time (typically in s). The power spectral density (interchangeably referred to as “power spectrum”) reflects the “frequency content” of the EEG signal, or the distribution of signal power over a frequency range. A power spectrum shows a signal that is a magnitude (or power, typically in μV²/Hz or dB/Hz where “dB” refers to decibel i.e. 10*log 10(μV²) or μV²/oct or dB/oct where “oct” refers to “octave” i.e. log₂(Hz)) at each of a plurality of frequencies (typically in Hz), obtained by decomposition of the original EEG signal in the frequency domain (e.g. using a Fourier transform). Spectral analysis (also referred to as spectral power analysis or power spectrum analysis) is commonly used to quantify the signals present in EEG or MEG, and in particular to detect the oscillatory electrical activity that characterises brain activity as measured with electroencephalography (EEG) or MEG. Spectral analysis assumes that an EEG or MEG signal is a linear combination of oscillatory processes at a plurality of respective specific frequencies, and decomposes the signal into frequency components, quantifying a signal that indicates the magnitude (or power) of the signal contributed by each frequency component. For example, a peak at a particular frequency in the power spectrum indicates the presence of an oscillation with the particular frequency in the original EEG/MEG data, with the magnitude of the peak in the power spectrum indicative of the amplitude of the oscillation in the original EEG/MEG data. While the frequency decomposition is not able to fully disentangle all the different brain processes, it may still serve that purpose in specific applications. Similarly, magnetoencephalography (MEG) is a method to record the magnetic fields resulting from ionic currents within the neurons of the brain. Thus, both EEG and MEG record signals indicative of neuronal activity. Further, both can be used to study oscillatory activity in the brain, following a similar principle of analysing signal at one or more frequencies. EEG and MEG can be used alone or in combination. The frequency range may be somewhat arbitrarily divided in subranges called “bands”. While there is not consensus of the exact definition an example of band definitions is given here: delta (δ) (0.2-4 Hz), theta (θ) (4-8 Hz), alpha (α) (8-12 Hz), beta (φ (12˜30 Hz) and gamma (g) (>30 Hz). Embodiments described herein relate in particular to the use of MEG/EEG signal in the beta band of frequency. While there is no fixed, universally accepted definition of the range encompassed by the beta-band, a general consensus encompasses the frequencies between 12 and 30 Hz, or the majority of this range. For example, in embodiments a range of 16 to 32 Hz is used. Such a range advantageously encompasses 1 octave. Such a range further advantageously captures the majority of the signal associated with GABA-A modulators. Other ranges are possible and explicitly envisaged, including ranges that combine the above boundaries (e.g. 16-30 Hz, 12-32 Hz) and ranges that are e.g. up to 10% narrower or wider than these rages by inclusion of frequencies are either one or both ends of the above ranges (e.g. any range within the 12-32 Hz range, any range within the 12+1.2 Hz to 32+3.2 Hz range, or any range that is at least one 12, 14, or 16 Hz units wide within these ranges). EEG/MEG power spectra (also referred to as power spectral density) comprise signals indicative of brain activity, and signal associated with background signal (also referred to simply as “background”). Background or background signal refers to a portion of a signal (e.g. magnitude of the power at each of a plurality of frequencies in a power spectrum) that is due to extraneous signal that is not indicative of a phenomenon to be measured. This may also be referred to as “noise”. This background signal may well reflect activity of the brain but reflects other processes not of interest that hamper the quantification of the process of interest.

The EEG signals are highly inheritable and therefore characteristic for an individual. Neuropsychiatric disorders can have characteristic EEG signatures that can manifest in frequency-specific changes in EEG power (for example, Dup15q syndrome is associated with excess oscillations in the beta-band (˜12-30 Hz) that are linked to GABA-A receptor abnormalities). Individual characteristics that are unrelated to e.g. a pathophysiology of interest can therefore lead to considerable variability, which prevent or decrease the reliability and/or applicability of use of EEG spectral power as a biomarker for a specific pathophysiology.

### Autism Spectrum Disorder (ASD)

Autism spectrum disorder (ASD) is a neurodevelopmental condition characterized by core symptoms of persistent deficits in social communication and social interaction, and the presence of restricted, repetitive patterns of behaviours, interests, or activities (American Psychiatric Association, 2013). ASD is likely an umbrella terms for different neurodevelopmental disorders (NDDs) with shared phenotypes but different underlying pathophysiologies. Thus the development of effective treatment for ASD will likely require the identification of subgroups of patients with more homogenous pathophysiology that may be responsive to drugs with a specific MoA.

There are several lines of evidence for GABAergic dysfunction and more specifically for GABA-A receptor dysfunction in ASD that could underlie symptoms in a subgroup of ASD. Neurodevelopmental disorder (NDD) risk genes are disproportionately associated with GABA function. ASD risk genes are enriched for GABA function, more expressed in interneurons, more correlated with genes overexpressed in interneurons (inhibitor neurons), and more likely to target proteins upregulated in interneurons (Wang et al 2018). Furthermore, rare genetic NDDs with symptomatic overlap to ASD often show impairment of GABAergic signalling. For example, these NDDs include: Angelman syndrome (Judson et al., 2016), Fragile X syndrome (Telias 2019), Dup15q syndrome, Rett syndrome (Chao et al., 2010), Neurofibromatosis Type 1 (NF1, Violante et al., 2016), and SLC6A1-related NDD (Goodspeed et al., 2020). Additionally, there is evidence for a reduced interneuron (inhibitory) cell count in ASD (Contractor, Ethell & Portera-Cailliau, 2021). Furthermore, the functional excitation/inhibition (E/I) ratio detectable in EEG recordings was found to be lowered by pharmacological enhancement of GABAA receptor-mediated inhibition, and E/I imbalance was shown to be present in ASD (Bruining et al., 2020; Robertson, Ratai and Kanwisher, 2015).

There is further evidence that GABA-A α5 receptor dysfunction in particular are involved in at least some ASD patients. As explained further below, the α5, β3 and γ2/γ3 GABA-A receptor subunits assemble to form so called GABA-A α5 receptors. The density of these receptors is maximal in regions implicated in ASD symptoms. Although this receptor subtype accounts for only approx. 5% of all GABA-A receptors, it account for up to 25% of GABA- in key brain areas implicated in ASD (including Hippocampus, Insular cortex, Nucleus Acumens, Medial prefrontal cortex). Further, mutations of the GABRB3 and GABRG3 have been linked to ASD, and GABRB3 and GABRA5 mutations are linked to early onset epileptic encephalopathies (EOEE, epilepsy being more frequent in individuals with ASD than in the general population). For example, copy number variations in the 15q11-13 chromosomal region, containing GABRB3, GABRA5, and GABRG3 occur in genetic diseases which result in symptoms that overlap with autism spectrum disorders and would therefore be classified as having an ASD in the absence of a more precise genetic diagnostic (e.g. approximately ˜60% of patients with Angelman Syndrome which have a deletion in this region would be classified as having an ASD versus on ˜20% that have AS due to UBE3A mutations), approximately 70% of patients with Dup15q would be classified as having an ASD due to symptomatic overlap to ASD). Finally, dysfunction in GABA-A α5 receptors is associated with restricted and repetitive behaviours in model animals, which are a hallmark of ASD. In particular, deletion of the gene for the α5 subunit of the GABA-A receptor (monogenic Gabra5−/− mice) caused robust autism-like behaviours in mice, including reduced social contacts and vocalizations (Zureck et al., 2016). Further, MP-III-022, a positive allosteric modulator (PAM) selective for GABA-A α5 receptors, prevented impairments in many parameters connected with social, repetitive and restrictive behavioural domains in Wistar rats prenatally exposed to valproic acid, as an animal model used for studying ASD (Santrac et al., 2022).

Thus, the present inventors have recognised that Angelman syndrome (associated with a deletion of the chromosomal region 15q11-13) and Dup15q syndrome (associated with copy number gains of the chromosomal region 15q11-13), two genetic neurodevelopmental disorders with a substantial degree of overlap with ASD (approx. 60% and 70% of individual meeting diagnostic criterial for ASD) represent examples of subpopulations of individuals that would have been diagnosed as having an ASD in the absence of a more precise diagnosis. The present inventors have further recognised that these subpopulations are associated with opposite genetic causes and opposite associated GABAergic dysfunction despite being both clinically similar to ASD. Thus, the present inventors have hypothesised that identification of individuals with ASD that have GABAergic dysfunction leading to GABAergic brain activity phenotypes similar to individuals with Angelman syndrome or Dup15q syndrome could be identified (even if these phenotypes may not be caused by the same genetic deficit and may have very different aetiology that may not involve the exact same receptors but converge at a circuit level to produce a similar brain activity phenotype), thereby enabling the selection of drugs with a mode of action consistent with the expected GABAergic dysfunction present in these individuals.

### Dup15q Syndrome

Dup15q syndrome is rare, genetic neurodevelopmental disorder characterized by a distinctive but heterogeneous neurodevelopmental phenotype. The birth incidence is estimated between 1 in 20,000 to 1 in 11,000 but data is limited. Hypotonia in infancy and global developmental delay (which includes motor, cognitive and language development) are the most frequent features (DiStefano et al., 2016; Kalsner and Chamberlain, 2015; Lusk et al., 1993). ASD, epilepsy and behavioral difficulties are common in Dup15q syndrome. Individuals with epilepsy usually demonstrate greater severity in all domains (DiStefano et al., 2020). Dup15q syndrome results from additional maternal copies of the 15q11-q13 region. The majority (˜80%) and most severely affected individuals present with an isodicentric (IDIC) supernumerary chromosome of maternal origin, resulting in triplication of the chromosomal segment (4 copies). The remaining 20% of the population have interstitial tandem rearrangements (INT), resulting in duplication (3 copies) of the 15q11.2-q13.1 region. Other, uncommon variations of these primary mechanisms include interstitial triplications, asymmetric copy number gains, as well as ultra-rare isodicentric hexasomies (or more). Paternally derived interstitial duplications are reported to cause a different, although genetically related disorder, presenting with a variable and mild neurodevelopmental phenotype.

The Prader-Will Angelman critical region (PWACR), a 5 Mb-long segment between BP2 and BP3 at 15q11.2-q13.1, is the minimum and critical chromosomal segment causal for Dup15q syndrome and contains several genes and non-coding regions. Notably, individuals with Dup15q syndrome can have larger duplications extending more proximal and distal. There is strong evidence suggesting gene-dose effects in this region: Patients with triplications are typically more severely affected than those with duplications whereas maternal deletions cause Angelman syndrome (AS). Copy number variations at this locus are thus believed to lead to altered expression of the encoded proteins resulting in abnormal function that, in turn, alter the developmental trajectory. Due to genomic imprinting in this region, gene expression patterns differ depending on the parental origin of the copy number gain. In Dup15q syndrome, defined by copy number gains of maternal origin, excess gene dosage in the PWACR is limited to UBE3A (the only paternally imprinted gene) and to non-imprinted, bi-allelically expressed genes including ATP10, OCA2, HERC2 and a cluster of three GABA-A receptor genes, GABRB3, GABRA5, and GABRG3. The genes involved in the etiology of Dup15q syndrome and their relative contributions remains elusive.

UBE3A, the only paternally imprinted gene on 15q11.2-q13.1, is considered a contributor to the pathophysiology of Dup15q syndrome (maternal copy number gains) given the more severe clinical phenotype compared to paternal duplications, and also given its causative role in Angelman syndrome. Recently, maternal UBE3A mutations that result in increased UBE3A activity have been identified and are associated with global developmental delay, and in some cases, with hypotonia or epilepsy. These findings indicate that an additional UBE3A, or increased UBE3A activity, are sufficient to cause neurodevelopmental syndromes and that UBE3A plays a role in the etiology of Dup15q syndrome. There are several lines of evidence that other duplicated genes also contribute to the etiology of Dup15q syndrome. First, UBE3A duplication and gain of function mutations do not fully recapitulate the Dup15q syndrome phenotype. Second, ultra-rare paternal interstitial triplications appear to manifest with the core clinical features of Dup15q syndrome and display moderate to severe deficits, comparable to maternal triplications. Third, in Angelman syndrome, individuals with deletion of maternal 15q11.2-q13.1 are substantially more impacted than individuals with mutations disrupting expression of functional UBE3A. Furthermore, gain of function mutations of the bi-allelically expressed genes on 15q11.2-q13.1 also cause neurodevelopmental disorders.

Given the important role in brain development and function, the 15q11.2-q13.1 GABA-A receptor subunits genes are considered as other potential pathogenic elements for Dup15q syndrome (Hogart et al., 2010; Lusk et al., 1993). Moreover, GABRB3 is an established risk gene in ASD (Sanders et al., 2015) and mutations in 15q11.2-q13.1 GABA-A receptor subunit genes have been linked to multiple neurodevelopmental disorders. Mutations in either GABRB3 or GABRA5 genes cause epileptic encephalopathies. Both gain-of-function and loss-of-function GABRB3 variants have been described and shown that patients with mutations that increase GABAergic activity have more severe phenotypes (Absalom et al., 2020). The gene products of the three GABA-A receptor subunit genes on 15q11.2-q13.1, the β3, α5, and γ3 subunits of GABA-A receptors, co-assemble to form a functional GABA-A α5 receptor subtype. The present inventors therefore postulated that increased GABA-A α5 receptor expression leading to increased GABA-A signaling is part of the Dup15q pathophysiology.

Individuals with Dup15 syndrome have a distinctive EEG phenotype characterized by excess beta-band (12-30 Hz) oscillations (Frohlich et al., 2016; Lusk et al., 1993; Urraca et al., 2013). For example, more than 100% power increase (i.e., more than 2 fold) compared to typically developing controls has been reported in individuals with Dup15 syndrome (Frohlich et al., 2019a). Several lines of evidence indicate that this signature reflects excess GABA-A receptor function related to the copy number gains of the 15q11.2-q13.1 GABA-A receptor subunit gene cluster. First, oscillations in the beta-band are a well-known phenomenon induced by GABA-A enhancing drugs including benzodiazepines (Greenblatt et al., 1989) and in particular for GABA-A a5 positive allosteric modulators (such as e.g. Alogabat, see below). Second, excess beta-band oscillations are also present in patients with paternal duplications, suggesting a link to non-imprinted genes (Frohlich et al., 2019a). The opposite EEG phenotype (i.e., decreased beta-band power) is found for individuals with AS with deletion genotype compared to individuals with AS caused by UBE3A mutations (Frohlich et al., 2019b), again suggesting a role of non-imprinted genes. Thus, EEG beta-band oscillations can be considered a biomarker to quantify excess GABA-A function in Dup15q syndrome.

### Angelman Syndrome

Angelman syndrome (AS) is a rare genetic neurodevelopmental disorder with a prevalence of 1 in 10,000 to 24,000 births. Clinical traits of AS include global developmental delay, intellectual disability, microcephaly, epilepsy, sleep difficulties, and some phenotypic overlap with autism (see e.g. Trillingsgaard A, Østergaard JR (2004),Williams 1995, Thibert et al. 2013, Bird 2014).

The aetiology of AS can be divided into two broad groups. The first group, nondeletion AS, primarily affects the function or expression of UBE3A. Nondeletion AS, comprising 25% to 30% of all AS cases, includes UBE3A mutations, imprinting defects, and paternal uniparental disomy. The second group, deletion AS, is defined by deletions of the maternal chromosome 15 at 15q11.2-q13.1. Deletions vary in length but all encompass UBE3A, as well as about 11 to 15 other protein-coding genes, numerous small nucleolar RNA genes, and noncoding regions of potential functional significance. Deletion AS accounts for the majority (about 70%) of AS cases. Individuals with deletion AS have a more severe clinical presentation than those with nondeletion AS, suggesting that deletion of genes other than UBE3A add to disease severity (Keute et al., 2020). Deletions of 15q11.2-q13.1 include the GABRB3-GABRA5-GABRG3 gene cluster, which encodes the β3, α5, and γ3 gammaaminobutyric acid type A (GABA-A) receptor subunits, which co-assemble to form a functional GABA-A α5 receptor subtype. Given the important role of the GABAergic system in brain development and function, the deleted GABA-A receptor subunit genes may cause important differences in AS genotypes. Deletion AS compared to non-deletion AS is characterized by a decrease in beta-band power (Frohlich et al., 2019b), which resembles the pharmacodynamic effect obtained by GABA-A α5 receptor negative allosteric modulation (Hipp et al., 2021). Thus, this EEG signature is thought to reflect reduced GABA-A α5 receptor function due to the deletion of the GABA genes on 15q11.2-q13.

### GABA Function and Modulators

GABAergic interneurons are neurons that release the neurotransmitter gammaaminobutyric acid (GABA). Interneurons are neurons that primarily function to connect other types of neurons. The majority of interneurons are inhibitory interneurons, including GABAergic interneurons. GABAergic neurons release GABA to (negatively) regulate the firing rate of target neurons. GABAergic neurons are key for synchronization of neurons and dynamic processes in the brain. A key mediatory of inhibitory signalling are postsynaptic GABA_{A} receptors (also referred to herein as “GABA-A” or “GABA_{A}” or “GABA_{A}” or “GABA a” receptors), a family of ligand-gated ion channels which respond to GABA. There are 19 genes encoding for GABA_{A} receptor subunits that assemble as pentamers, most commonly as two α, two μ, and one γ subunit. α5, β3 and γ2/γ3 subunits assemble to form GABA_{A} α5 receptors, which constitute about 5% of all GABA-A receptors in the brain. Most abundant are α1 containing receptors (Rudolph and Knoflach 2011).

GABA has two allosteric binding sites at the interface of the α and γ subunits. Many compounds in clinical use as anxiolytics, sedatives, hypnotics or antiepileptics bind to the allosteric benzodiazepine (BZD) binding site which is formed by one of the a subunits (α1, α2, α3 or α5) and usually the γ2 subunit. Others such as barbiturates bind to the barbiturate binding site, possibly at β subunits. GABA also has allosteric binding sites that are the targets of neuroactive steroids. All of these compounds are known as positive allosteric modulators (PAMs) since they have no effect alone, but increase the activity of GABA-A receptors in the presence of GABA. In contrast, negative allosteric modulators (NAMs) decrease the activity of GABA-A receptors. Non-selective GABA-A NAMs are of very limited clinical utility as they cause seizures and selectivity to e.g. GABA-A a5 (or GABA-A a2, a3 and/or a5) is typically necessary (Hipp et al.,2021). GABA-A receptor enhancing drugs (also referred to herein as GABA-A activators or positive modulators) include Benzodiazeipines (which bind to the BZD binding site), Barbiturates (which bind to the barbiturate binding site) and neuroactive steroids (in particular, inhibitory neurosteroids such as tetrahydrodeoxycorticosterone (THDOC), the androstane 3α-androstanediol, cholestane and allopregnanolone (3α, 5α-THP, also known as brexanolone, CAS516-54-1, and sold as Zulresso), zurranolone (also known as SAGE-217, CAS 1632051-40-1) and ganoloxone (CAS 38398-32-2)). These drugs have been shown to increase the EEG power signal in the beta-band (Schomer and Silva, 2012). Indeed, non-selective GABA-A PAMs such as benzodiazepines, are known to induce a characteristic EEG signature with an increase in beta-band activity which is an established pharmacodynamic marker, along with a decrease of activity at lower frequencies (Friedman et al., 1992; Malizia et al., 1996; Visser et al., 2003). Similarly, non-selective GABA-A NAMs have been shown to decrease beta-band power (e.g., DMCM, Ro 19-4603) (Visser et al., 2003). Selective drugs exist that only modulate a specific subtype or set of subtypes of GABA-A receptors. For example, drugs that only modulate α1 containing GABA-A receptors include zolpidem, zopiclone, eszopiclone, zalpion and zolpidem (collectively known as “z-drugs”). Drugs that only modulate α2/α3 containing GABA-A receptors, and drugs that only modulate α5 containing GABA-A receptors are also known (as described further below). Each of these selective GABA-A modulators are known to have some effect on EEG beta power. Thus, as described herein, a GABA-A modulator may be a selective GABA-A modulator, a GABA-A α5 modulator, a GABA-A α2/α3 modulator, or a GABA-A α2/α3/α5 modulator. Examples of GABA-A α2/α3/α5 modulators include PF-06372865 (CAS 1614245-70-3). Examples of GABA-A α2/α3 modulators include the neurosteroids allopregnanolone/brexanolone (CAS516-54-1, sold as Zulresso), zurranolone/SAGE-217 (CAS 1632051-40-1) and ganoloxone (CAS 38398-32-2).

GABA-A receptors containing the α5 subunit are referred to as GABA-A α5, GABA-A a5, GABA α5, GABA a5, α5GABA_{A}, a5GABA_{A}, GABAα5, or GABAa5 receptors. They are typically composed of β3, α5, and γ2/3 subunits. Both genetic and pharmacological studies have demonstrated that GABA-A α5 subunit-containing receptors play an important modulatory role in learning and memory processes, in line with their preferential expression in the hippocampus and other key cortical regions of the limbic system. Compounds with selectivity for the α5 containing receptors do not suffer the same side effects as non-selective NAMs (including anxiogenic or proconvulsive side effects) and PAMs (including sedation).

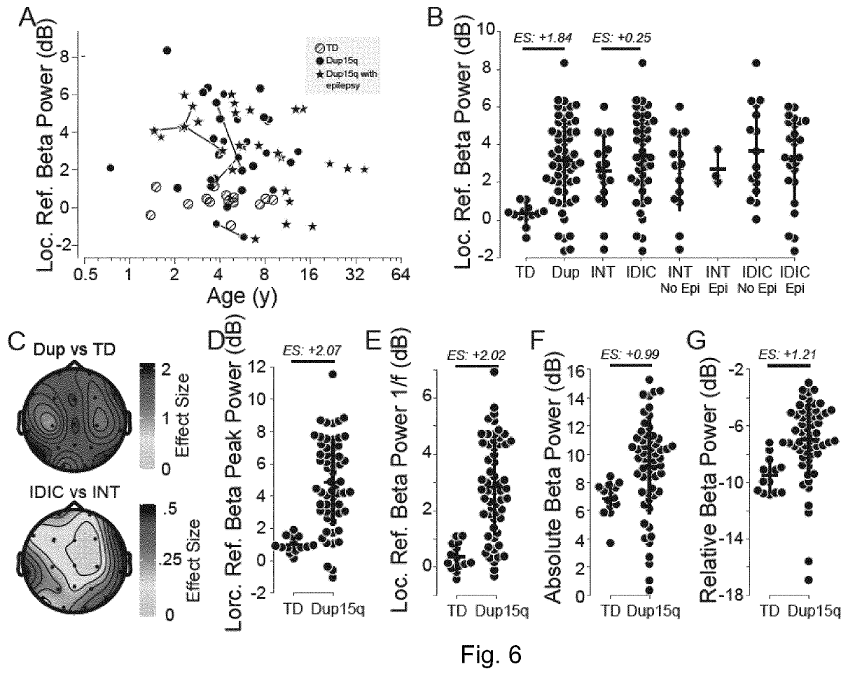

GABA-A α5 repressors (including NAMs) hold promise as potential treatments for multiple indications, particularly associated with cognitive impairment. For example, treatment of Alzheimer's disease, mild cognitive impairment, cognitive impairment associated with Down Syndrome (DS), cognitive impairment associated with schizophrenia (CIAS), and cognitive impairment after anaesthesia have been suggested. A GABA-A α5 repressor may be a negative allosteric modulator. A GABA-A α5 NAM may be selected from basmisanil (CAS 1159600-41-5), RO15-4513 (CHEMBL6597, Ethyl-8-azido-5,6-dihydro-5-methyl-6-oxo-4H-imidazo-1,4-benzodiazepine-3-carboxylate), L-655708 (CHEMBL52030), RY-080 (Skolnick et al., 1997), PWZ-029 (CHEMBL45246, -chloro-3-(methoxymethyl)-5-methyl-4H-imidazo[1,5-a][1,4]benzodiazepin-6-one), RO4882224 (Knust et al., 2009), RO4938581 (Knust et al., 2009), any compounds described in US 2015/0152085 (4-(2-Fluorophenyl)-1-methyl-1H-1,2,3-triazole-5-carbaldehyde), alpha5lA (Sternfeld et al., 2004), alpha5lA-II (Sternfeld et al., 2004), MRK-016 (CAS342652-67-9)), MRK-536 (Chambers et al., 2003), ZK-93426 (CAS 1216792-30-1), Compound 2 (Liu et al., 2010), and ONO-8590580 (CAS 1802661-73-9). Basmisanil has already been assessed in Phase II clinical studies for intellectual disability in DS (https://clinicaltrials.gov/ct2/show/NCT02024789) and CIAS (https://clinicaltrials.gov/ct2/show/NCT02953639). Basmisanil has been previously shown to decrease EEG beta-band power in healthy volunteers (see Hipp et al., 2021, FIG. 6). A GABA-A α5 repressor may be a GABA-A α5 antagonist, such as e.g. afizagabar (CAS 1398496-82-6). The GABA-A α5 repressor may be a NAM, preferably Basmisanil, or an antagonist such as afizagabar.

A GABAA-α5 activator may be a positive allosteric modulator (PAM). A GABA-A α5 activator may be selected from MP-III-022, alogabat (CAS 2230009-48-8), any of the compounds described in WO 2021/198124, any of the compounds described in WO2019/246300 and WO2021/127543, Compound 6 (methyl 3,5-diphenylpyridazine-4-carboxylate, van niel et al., 2005), compound 44 (6,6-dimethyl-3-(3-hydroxypropyl)thio-1-(thiazol-2-yl)-6,7-dihydro-2-benzothiophen-4(5H)-one, Chambers et al., 2003), SH-053-R—CH3-2′F (ethyl (4R)-8-ethynyl-6-(2-fluorophenyl)-4-methyl-4H-imidazo[1,5-a][1,4]benzodiazepine-3-carboxylate, CHEMBL4571892), SH-053-2′F—S—CH₃ (Savic et al., 2008, S53d-acid (Simeone et al., 2021), MP-III-022 (Simeone et al., 2021), PF-06372865 (CAS 1614245-70-3), GL-LL-73 ((4R)-8-ethynyl-6-(2-fluorophenyl)-N,N,4-trimethyl-4H-imidazo[1,5-a][1,4]benzodiazepine-3-carboxamide, CHEMBL4537552), GL-II-74 (van Amerongen et al., 2019) and GL-II-75 (Prevot et al., 2019). Preferably, the GABA-A α5 activator is alogabat. As shown further below, the present inventors have shown that alogabat increases EEG beta-band power in healthy volunteers.

Thus, the present inventors have shown that the effect of positive and negative modulators of GABA-A α5 can be seen in the beta-band of EEG power in healthy subjects. They have further shown that individuals with GABA-A loss or gain of function through genetic alterations show corresponding signals in the beta-band of EEG power, with respectively a decreased (for individuals with loss of GABA-A function) or increased (for individuals with gain of GABA-A function) signal in the beta-band compared to healthy subject. The present inventors have further developed a sensitive method to detect this loss or gain of function by analysis of the EEG beta-band power, and thus to stratify patients according to their GABA phenotype, select patients for treatment with GABA-A modulators (i.e. usable as a companion diagnostic to any such therapy), select patients for clinical trials of drugs for treatment of conditions associated with GABA-A dysfunction, and monitor treatment of patients with drugs for the treatment of conditions associated with GABA-A dysfunction.

Many neuropsychiatric disorders have been associated with GABA dysfunction (also referred to herein as GABAergic dysfunction), and in particular GABA-A dysfunction. The present disclosure applies to any disease, disorder or condition that is associated with a GABAergic dysfunction, collectively referred to as neuropsychiatric disorders associated with a GABAergic dysfunction. For example, evidence of GABAergic dysfunction has been found in most neuropsychiatric disorders, including e.g. Alzheimer's disease and Schizophrenia. Conditions associated with GABA-A dysfunction include neurodevelopmental disorders such as ASD. In particular, many genetic neurodevelopmental disorders with some phenotypic overlap with ASD have been associated with GABA-A dysfunction including Angelman syndrome, Fragile X syndrome, Dup15q syndrome, Rett syndrome, NF1, SLC6A1. Each of these diseases or disorders may be referred to as conditions associated with GABA dysfunction.

### Determination of Neurological Dysfunction

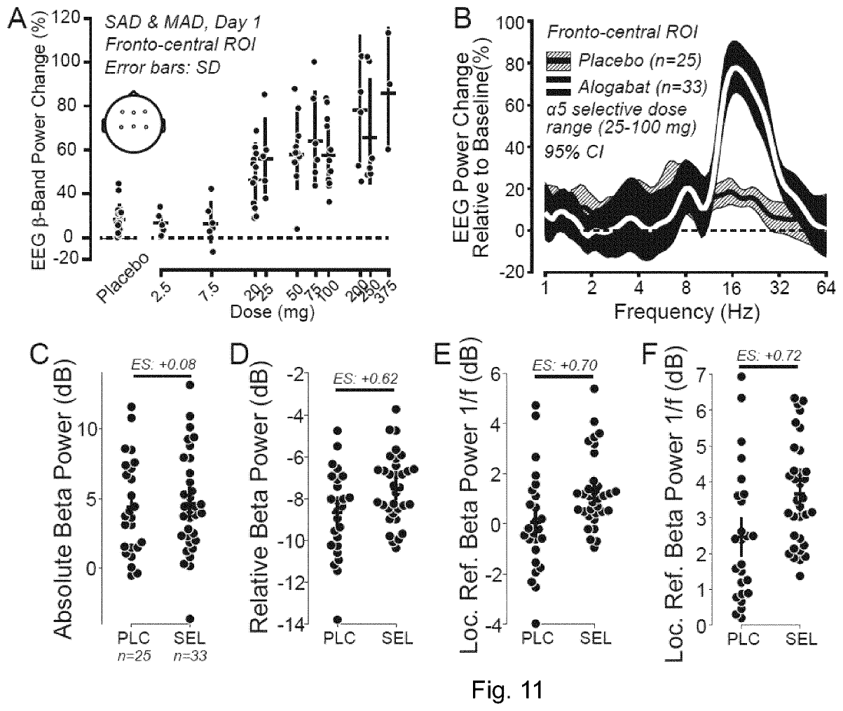

In embodiments of the present invention, a prediction of neurological functional status, such as a GABAergic functional status or GABA-A functional status is performed. In these embodiments, this prediction is performed by a computer-implemented method or tool that takes as its inputs EEG data (or MEG data) from the patient, and produces as output an indication of whether a subject has a particular neurological dysfunction, such as a GABA or GABA-A dysfunction. Prior to the present invention, although a link between the effect of GABA-A modulating drugs, AS and Dup15q and EEG power in the beta-band was known, EEG data and in particular the EEG power signal in the beta frequency band was not recognised, known or proposed as a biomarker of GABA-A dysfunction or criterion for patient selection for GABA-A modulation. This may be at least in part because any signal in the EEG power spectrum that may have been sufficiently informative for this purpose could not be quantified reliably enough for this to be used as a biomarker of GABA-A dysfunction or GABA-A modulation. For example, prior work failed to establish a relationship between EEG beta-band power as a proxy of excess GABA-A function and symptom severity in Dup15q syndrome (see Saravanapandian et al., 2020). The present inventors have demonstrated that this relationship could not be observed not because excess GABA-A function, as reflected in increased EEG beta oscillations, was not an important contributor to the Dup15q pathophysiology, but because prior approaches to quantify the excess EEG beta-band oscillations in Dup15q were not specific and sensitive enough. As a consequence, a relationship with symptom severity could not be established using prior art approaches, whereas not only is this possible with the methods of the present disclosure, the procedure described in the present disclosure provides a much greater separation between typical developing controls and individuals with Dup15q syndrome (resulting in a much more sensitive biomarker). Additionally, as further demonstrated in the examples below, prior art metrics like absolute beta power are not sensitive enough to detect GABA-A drug effects in parallel group designs, while the procedure described in the present invention allows to do so (see e.g. FIGS. 11-C, E, F as described further below). Another advantage of the methods described herein is that they can be used to identify a decrease in a particular frequency range (e.g. beta-band power), which cannot be identified using e.g. absolute power.

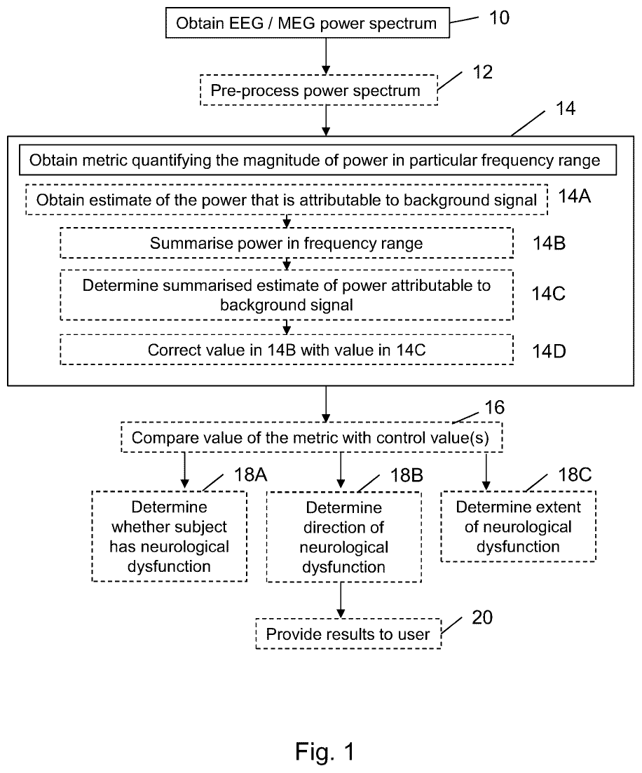

FIG. 1 is a flow diagram showing, in schematic form, a method of characterising a subject as having a neurological dysfunction, such as a GABA-A dysfunction according to the disclosure. At step 10, an EEG and/or MEG power spectrum is obtained from a subject. This may comprise one or more optional steps selected from: obtaining an EEG/MEG recording from a subject, performing spectral analysis to obtain one or more power spectra from the EEG/MEG recording, combining a plurality of power spectra such as e.g. by averaging. Optionally, a one or more reference (also referred to as “control”) EEG and/or MEG power spectra may also be obtained from one or more control subjects, such as e.g. healthy individuals (also referred to as “typically developing subjects”), the same subject prior to treatment, one or more subjects having no received a treatment (control or placebo group), etc. At optional step 12, the power spectrum may be pre-processed, for example to remove artefacts. Methods of pre-processing EEG/MEG power spectra are known in the art. At step 14, a metric quantifying the magnitude of the power in a particular frequency range is determined. This may comprise obtaining an estimate of the power in said frequency range that is attributable to background signal. Such an estimate may be obtained as a plurality of points (also referred to herein as anchor points), or as a relationship between frequency and power in said frequency range (i.e. a curve). Such a curve can be obtained using the plurality of anchor points (e.g. by curve fitting, e.g. linear curve fitting) or by fitting the power spectrum with a superposition of oscillatory signals and a 1/f^{α} component (e.g. as explained in Donoghue et al. 2020), and extracting the 1/f^{α} component of this global fit of in said frequency range. Anchor points are points that are outside of said frequency range or on the boundaries thereof, and that are selected based on proximity to the frequency range and likelihood of capturing background signal (i.e. points unlikely to reflect other bona fide brain activity). At step 14B, the power in said frequency range may be summarised, for example by selecting a value of the power in said frequency range, obtaining a value as an average or weighted average of a plurality of values, or integrating the power curve in said frequency range. At step 14C, the estimate of power in said frequency range attributable to background signal may be summarised, for example by determining a value using the plurality of anchor points (e.g. as an average or weighted average of the plurality of anchor points), or by integrating the curve in the frequency range. At step 14D, the value obtained at step 14B may be corrected using the value obtained at step 14C. For example, the value of step 14C may be subtracted from the value of step 14B, or the value of step 14B may be divided by the value of step 14C (all optionally after log transformation). At optional step 16, the value of the metric obtained at step 14 may be compared to one or more control values, such as e.g. values obtained by applying the process of steps 14 and optionally 12 to one or more control/reference spectra. This may be used to determine whether the subject has a neurological dysfunction at step 18A, for example if the value obtained at step 14 is significantly different from a control value or set of values. Instead or in addition to this, this may be used to determine the direction of a neurological dysfunction of the subject, for example based on whether the value obtained at step 14 is significantly lower or higher than a control value or set of values. Instead or in addition to this, this may be used to determine the extent of a neurological dysfunction of the subject, for example based on the magnitude of the difference between the value obtained at step 14 and a control value or set of values.

### Uses of Determination

A prediction of whether a subject is likely to have a neurological dysfunction, such as e.g. a GABA or GABA-A deficiency or excessive -A function, can be used in the treatment of a neuropsychiatric disorder. Thus, the invention also provides a method of treating a neuropsychiatric disorder in a subject, wherein the method comprises selecting, administering or recommending a subject for administration of a particular therapy or selecting, administering or recommending a subject for administration of an alternative therapy (including the selection or recommendation that a particular therapy is not given), depending on whether the subject is identified as likely to be have a particular neurological dysfunction, such as a GABA or GABA-A dysfunction, and/or depending on whether the subject is identified as likely to have a GABA-A excessive or defective function.

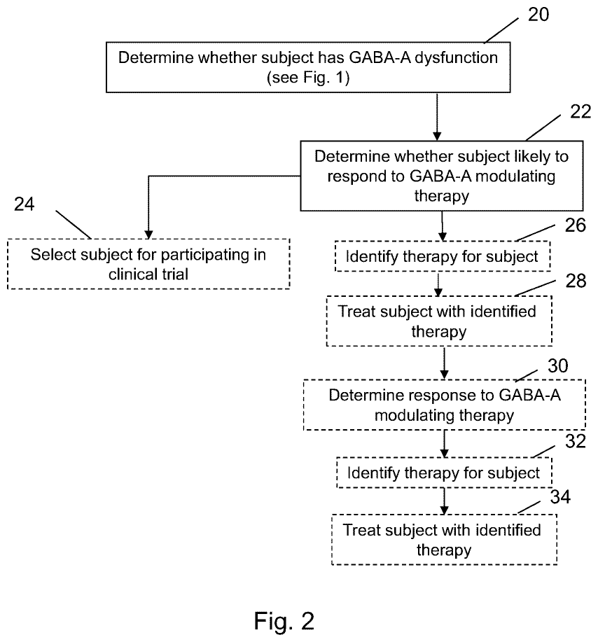

FIG. 2 illustrates a method of providing a treatment recommendation, selecting a subject for participating in a clinical trial and/or treating a subject that has been diagnosed as having a neuropsychiatric disorder or being likely to have a neuropsychiatric disorder, according to embodiments described herein. FIG. 2 illustrates in particular an embodiment in which a subject is characterised in relation to GABA-A dysfunction. However, other neurological dysfunctions and corresponding treatments can be analysed in a similar way. At step 20, it is determined whether the subject has a GABA dysfunction, particularly a GABA-A dysfunction, using methods described herein such as e.g. by reference to FIG. 1. Based on this determination, the subject may be classified as being likely to respond to a negative or positive GABA-A modulation therapy at step 22. In particular, subjects with lower than expected or higher than expected metric as described herein (e.g. quantifying EEG beta-band power and comparing to corresponding control values or set of values) may be identified as having an increased likelihood of response to GABA-A modulating therapy. In other words, subjects with lower or higher than expected metric as described herein (e.g. quantifying EEG beta-band power) may be identified as likely to benefit from GABA-A modulation therapy. Instead or in addition to this, subjects may be identified as likely to benefit from GABA-A modulation therapy based on a departure from an expected metric as described herein (e.g. quantifying EEG beta-band power), where the likely benefit depends on the extent of the departure from the expected value of a metric as described herein (e.g. quantifying EEG beta-band power). At optional step 26, a particular course of treatment (which may comprise one or more different individual therapies) may be identified based on the results of step 22. For example, a subject that has been identified at step 22 as likely to respond to or benefit from a positive GABA-A modulation therapy may be identified as likely to respond to or benefit from a therapy that includes a positive GABA-A modulator, such as a GABA-A PAM. As another example, a subject that has been identified at step 22 as likely to respond to or benefit from a negative GABA-A modulation therapy may be identified as likely to respond to or benefit from a therapy that includes a negative GABA-A modulator, such as a GABA-A NAM. As another example, a subject that has been identified at step 22 as unlikely to respond to a GABA-A modulation therapy may be identified as likely to benefit from a therapy that is different from the particular course of therapy. Such a subject may be selected for or recommended for a course of therapy that does not include the particular course of therapy. The alternative course of therapy may be a specific alternative or may be a recommendation that the particular course of therapy is not used. Alternatively, a subject that has been identified at step 22 as likely to respond to the particular course of therapy may be identified as likely to benefit from a therapy that includes the particular course of therapy. At optional step 28, the subject may be treated with the therapy identified at step 26. At optional step 24, a subject may be selected for inclusion in a clinical trial for a particular course of treatment (which may comprise one or more different individual therapies), based on the results of step 22. For example, a subject that has been identified at step 22 as likely to respond to a positive GABA-A modulation therapy may be selected for inclusion in a clinical trial for a therapy that includes a positive GABA-A modulator, such as a GABA-A PAM. As another example, a subject that has been identified at step 36 as likely to respond to a negative GABA-A modulation therapy may be selected for inclusion in a clinical trial for a therapy that includes a negative GABA-A modulator, such as a GABA-A NAM. At optional step 30, the method of FIG. 1 is applied again using a power spectrum obtain after administration of a therapy (e.g. acute administration) and is used to monitor the subject, for example wherein the subject is undergoing a therapy for the neuropsychiatric disorder. For example, the result of step 30 may be compared with one or more previous results from the same subject (such as e.g. a result obtained at baseline and/or at a previous timepoint). The result of step 30 may also be used to make a treatment recommendation at step 32, for example by monitoring the response of the subject to administration of a particular therapy. Thus, a subject showing a positive response (e.g. a change in a metric as described herein in the expected direction, and optionally of a minimum magnitude, upon administration of a particular course of therapy, compared to the metric prior to administration of the particular course of therapy) may be selected for, recommended, or administered (optional step 34) a course of therapy that includes the particular therapy. Alternatively, a subject may be selected for, recommended, or administered a course of therapy that includes a particular therapy based on the value of a metric as described herein (e.g. quantifying EEG beta band power) quantified before (also referred to as “baseline”) and after administering the particular therapy. The measurement obtained after administering the particular therapy may be referred to as “acute” or “acute response” measurement if this is obtained after acute administration of the particular therapy. The selection, recommendation or administration (optional step 34) of the particular therapy may refer to the administration of the particular therapy in chronic manner, i.e. as a course of therapy comprising repeated administration. As another example, the result of step 30 may be compared with a suitable control result, such as e.g. a result from a control cohort comprising one or more healthy subjects and/or one or more subjects receiving a placebo treatment.

Thus, a method as described herein may comprise identifying whether a subject is likely to benefit from treatment with a particular course of therapy including chronic administration of a particular therapy (in this case, a GABA-A modulating therapy), the method comprising determining whether a subject has a GABA (in particular, GABA-A) dysfunction using a method as described herein after acute administration of the particular therapy. The method may further comprise determining whether a subject has a GABA (in particular, GABA-A) dysfunction using a method as described herein prior to administration of the particular therapy (i.e.at baseline). The method may comprise comparing or combining the results of the methods as described herein prior to and after administration of the particular therapy.

A method as described herein may comprise classifying the subject between a group that is likely to respond to a GABA-A modulating therapy (where a response as used herein refers to a positive response unless indicated otherwise), and a group that is not likely to respond to a GABA-A modulating therapy. Alternatively, a method as described herein may comprise classifying the subject between a group that is likely to respond to therapy with a GABA-A positive modulator, and a group that is not likely to respond to therapy with a GABA-A positive modulator. Alternatively, such a method may comprise classifying the subject between a group that is likely to respond to therapy with a GABA-A negative modulator, and a group that is not likely to respond to therapy with a GABA-A negative modulator. Alternatively, such a method may comprise classifying the subject between a group that is likely to respond to therapy with a GABA-A negative modulator, and a group that is likely to respond to therapy with a GABAA positive modulator. Alternatively, such a method may comprise classifying the subject between a group that is likely to respond to therapy with a GABA-A negative modulator, a group that is not likely to respond to therapy with a GABA-A modulator, and a group that is likely to respond to therapy with a GABA-A positive modulator. Alternatively, a method as described herein may comprise classifying the subject between a group that is likely to respond negatively to a GABA-A modulating therapy, and a group that is not likely to respond negatively to a GABA-A modulating therapy (step 22). In other words, a method as described herein may comprise classifying the subject between a group that is likely to experience detrimental effects in response to a GABA-A modulating therapy, and a group that is not likely to experience detrimental effects in response to a GABA-A modulating therapy. Alternatively, a method as described herein may comprise classifying the subject between a group that is likely to negatively respond to therapy with a GABA-A positive modulator, and a group that is not likely to negatively respond to therapy with a GABA-A positive modulator. Alternatively, such a method may comprise classifying the subject between a group that is likely to respond negatively to therapy with a GABA-A negative modulator, and a group that is not likely to respond negatively to therapy with a GABA-A negative modulator. Alternatively, such a method may comprise classifying the subject between a group that is likely to respond negatively to therapy with a GABA-A negative modulator, and a group that is likely to respond negatively to therapy with a GABAA positive modulator. Alternatively, such a method may comprise classifying the subject between a group that is likely to respond negatively to therapy with a GABA-A negative modulator, a group that is not likely to respond to therapy with a GABA-A modulator, and a group that is likely to respond negatively to therapy with a GABA-A positive modulator. This may be used to identify a therapy at step 26, which may optionally be administered to the subject at step 28.

A method as described herein may instead or in addition comprise determining the likely benefit of a course of treatment including a GABA-A modulating therapy for a subject, the method comprising determining whether a subject has a GABA (in particular, GABA-A) dysfunction using a method as described herein prior to and/or after acute administration of the particular therapy, wherein the magnitude of the metric as described herein (e.g. EEG beta-band power) quantified prior to and/or after acute administration of the particular therapy is indicative of the likely benefit of the course of treatment including a GABA-A modulating therapy. The GABA-A modulating therapy may be a GABA-A negative modulator or a GABA-A positive modulator. Reference to a metric having a value lower than expected or higher than expected may refer to the metric being significantly different from zero, or any value indicative of a difference between the signal and the estimate of the background signal as described herein. Reference to a metric having a value lower than expected or higher than expected may refer to the metric being significantly different from the value of the metric quantified for one or more control data sets, such as e.g. from EEG/MEG data for one or more control subjects.

In some cases GABA-A modulation therapy may comprise treatment with a GABA-A positive modulator, such as a PAM, or a GABA-A negative modulator, such as a NAM. A GABA-A positive modulator may be a non-selective GABA-A positive modulator, such as a Benzodiazeipine or a Barbiturate.A GABA-A negative modulator may be a selective GABA-A negative modulator, such as basmisanil. Advantageously, the GABA-A negative modulator may be an α5 selective GABAA negative modulator, such as basmisanil. A GABA-A positive modulator may be a selective GABA-A positive modulator, such as zolpidem, zopiclone, eszopiclone, zalpion, zolpidem and alogabat, advantageously a GABA-A α5 positive modulator, such as a GABA-A positive modulator selected from MP-III-022 and alogabat.

Any treatment described herein may be used alone or in combination with another treatment.

The subject is preferably a human patient. The subject may be a paediatric patient. The subject may be an adult patient. The subject may be a model animal. The model animal may be a mammalian, such as e.g. a mouse or a rat. The subject may be a neuronal cellular culture or brain organoid (including e.g. neuronal tissue or organoids derived from induced pluripotent stem cells). For example, a method as described herein may comprise making a determination for a subject (e.g. whether the subject has a neurological dysfunction, such as e.g. a GABA-A dysfunction) by applying the methods described herein to an electrophysiological recording obtained from a neuronal cell culture (including e.g. a neuronal tissue or brain organoid in culture) derived from the subject. The neuronal cell culture may have been derived previously from induced pluripotent cells (iPSCs) derived from cells of the subject. The methods described herein my comprise one or more of: obtaining cells from the subject, obtaining iPSCs from cells previously obtained from the subject, obtaining a neuronal cell culture from iPSCs previously obtained, obtaining an electrophysiological recording from the neuronal cell culture. Any system in which neurological electrophysiological signals (e.g. including electronic an/or magnetic oscillations associated with neuronal activity) can be recorded may be used within the context of the present invention. These may be referred to as electrophysiological recordings. In such cases, references to “EEG” or “MEG” may refer to intracellular recordings of neuronal activity.

### Systems

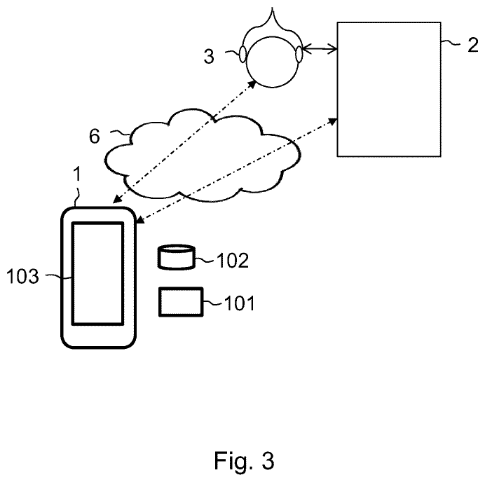

FIG. 3 shows an embodiment of a system for characterising subject (e.g. determining whether a subject has a neurological dysfunction, is likely to respond positively or negatively to a course of treatment, etc.) and/or for providing a treatment recommendation for a subject and/or for selecting a subject for participating in a clinical trial, and/or for monitoring a subject, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals, by producing a report, etc. The computing device 1 is communicably connected, such as e.g. through a network, to EEG/MEG data acquisition means 3, such as an electroencephalograph or magnetoencephalograph, and/or to one or more databases 2 storing EEG/MEG data. The EEG/MEG data may be raw or pre-processed EEG/MEG data. The one or more databases 2 may further store one or more of: control data, parameters (such as e.g. thresholds derived from control data, parameters used for normalisation, recording characteristics of the EEG amplifier such as sampling rate, high and lowpass filter frequencies or frequency response, electrode impedances, electrode locations or names, recording reference location, etc.), clinical and/or subject related information (such as e.g. age, sex, BMI, head circumference, etc.), etc. The computing device may be a smartphone, tablet, personal computer or other computing device. The computing device is configured to implement a method for characterising a subject, as described herein. In alternative embodiments, the computing device 1 is configured to communicate with a remote computing device (not shown), which is itself configured to implement a method as described herein. In such cases, the remote computing device may also be configured to send the result of the method to the computing device. Communication between the computing device 1 and the remote computing device may be through a wired or wireless connection, and may occur over a local or public network 6 such as e.g. over the public internet. The sequence data acquisition means may be in wired connection with the computing device 1, or may be able to communicate through a wireless connection, such as e.g. through WiFi and/or over the public internet, as illustrated. The connection between the computing device 1 and the EEG/MEG data acquisition means 3 may be direct or indirect (such as e.g. through a remote computer). The EEG/MEG data acquisition means 3 are configured to acquire EEG and/or MEG data from a subject, for example EEG data for one or more electrodes, and/or EEG data summarised (e.g. averaged) over a plurality of electrodes. In some embodiments, the EEG data be or may have been subject to one or more preprocessing steps.

The following is presented by way of example and is not to be construed as a limitation to the scope of the claims.

### EXAMPLES

The examples below illustrate the validity and utility of the methods of the present disclosure in the particular context of extracting measures of GABA-A dysfunction. In particular, Example 1 shows that in individuals with a genetic NDD implicating excess GABA-A function (Dup15q syndrome) the methods described herein provide a metric that can better separate this group form typical developing controls with presumed normal GABA-A function. Furthermore, the metric provided by a method as described herein shows a cross-sectional correlation with symptom severity, which provides evidence for the utility of quantifying GABA-A dysfunction, since such correlations were not identified previously with more common measures.

Example 2 shows that individuals with a genetic NDD implicating reduced GABA-A function (Angelman syndrome) the methods described herein provide a metric that can separate this group from typical developing controls with presumed normal GABA-A function. Importantly, this example shows that measure points in the opposite direction compared to Dup15q syndrome can be obtained, such that the methods described herein provide metrics that are sensitive to the directionality of the dysfunction identified.

Example 3 shows that for a drug that selectively enhances the function of GABA-A a5 receptors (GABA-A a5 PAM alogabat) the methods described herein provide a metric that can identify enhanced GABA-A function even in a parallel group design. While a within subject contrast (pre vs post drug contrast) is most efficient to quantify pharmacodynamics effects, this example shows the sensitivity of the metric to GABA-A modulation as needed to identify patients with GABA-A dysfunction.

### Example 1—Metrics as Described Herein are Indicative of GABA-A Dysfunction and Reveal Cross-Sectional Correlation with Symptom Severity in Dup15q Syndrome

This example provides evidence that excess GABA-A receptor function is a relevant part of Dup15q syndrome pathophysiology, and describes an analysis approach to isolate oscillatory activity from the background of the EEG power spectrum, i.e. “locally referenced power”. This metric is related to GABA-A function and in line with the aetiology of Dup15q syndrome suggest excess GABA-A function. This metric is applied to EEGs from paediatrics individuals with Dup15q syndrome and typical developing controls, demonstrating good separation between the groups and reveals a relationship with metrics of symptom severity in Dup15q syndrome.

### Methods

EEG Dataset. 59 EEGs were collected from 52 children (age<18 years) with Dup15q syndrome and 14 EEGs from 14 typical developing children. Data were pre-processed and interpolated to a 10/20 montage as described in Saravanapandian et al., 2020. Informed written consent from parents of participants prior to the start of all study activities was obtained. Participants taking antiepileptic medications that are known to enhance beta EEG oscillations (e.g., benzodiazepines) were excluded from analysis. Spontaneous EEGs in the wake state were analysed that were at least a few minutes of duration.

Dup15q syndrome symptoms were quantified using performance based assessment, i.e., direct assessment of the participant by a trained psychologist and by a semi-structured interview with the caregiver. Part of these data have been analysed previously (DiStefano et al., 2020), see this publication for more details. Here is a brief account of the assessments:
Cognitive ability. Cognitive development was assessed with either the Differential Abilities Scale-Second Edition (DAS-II; Elliot, 2007) or the Mullen Scales of Early Learning (MSEL; Mullen, 1995). The MSEL was used to assess participants who were under 68 months of age, and participants who were older but unable to achieve a basal score on the DAS-II. There is high convergent validity between the MSEL and the DAS-II, supporting the combination of assessments (Bishop, Guthrie, Coffing, & Lord, 2011; Farmer, Golden, & Thurm, 2016). Ratio scores for full-scale developmental quotient (FSDQ), non-verbal developmental quotient (NVDQ) and verbal developmental quotient (VDQ) were calculated for each child and based on division of the age-equivalent score by chronological age. Ratio scores were used to account for the scores of children who performed outside of the standardized norms for their chronological age. For children who were tested with the DAS-II, NVDQ and VDQ were calculated from the protocol-specific sub-scores. For children who were administered the MSEL, VDQ was calculated using the average of the Receptive Language and Expressive Language subscale scores, and NVDQ was calculated using the average of the Visual Reception and Fine Motor subscale scores (Akshoomoff, 2006).

Adaptive behavior. Adaptive behaviour was assessed via parent report, using the Vineland Adaptive Behavior Scale-2nd Edition (VABS-II; Sparrow, Balla & Cicchetti, 2005) or 3rd Edition (VABS-3; Sparrow, Cicchetti & Saulnier, 2016). The VABS is a semi-structured interview conducted with the parent and assesses 4 domains of adaptive behavior: 1) communication, 2) daily living skills, 3) socialization, and 4) motor skills. Standard scores were used to facilitate pooling scores across versions.

EEG processing. EEG data was processed as described in Frohlich et al., 2019a. In particular, EEG data were bandpass filtered 0.5 to 45 Hz (finite impulse response filter), then portions of the data containing gross artifacts, as well as bad channels, were identified by visual inspection and excluded from analysis. Independent component analysis was applied (Jung et al., 2000) to remove remaining artifacts [FastICA algorithm, Hyvarinen 1999]. Finally, rejected channels were interpolated and data were re-referenced to average.

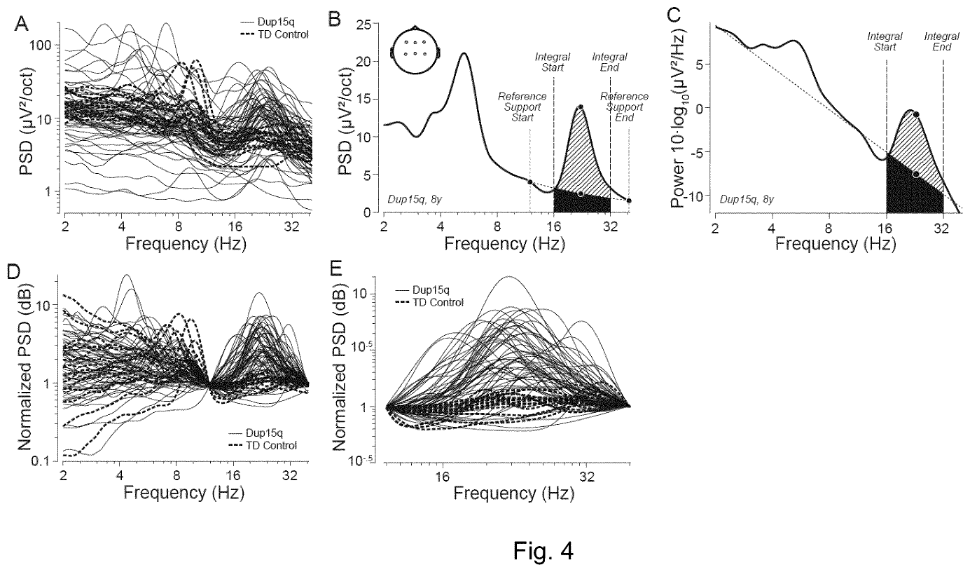

Extracting beta band oscillations. This example demonstrates a new analysis approach to isolate and quantify oscillatory neuronal activity from electrophysiological signals, which removes background signal using a local estimate of background. Here this approach is applied to isolate beta-band oscillations from EEG signals (FIGS. 4,5). This approach entails the estimation of the “background activity” in the frequency range of interest, which is non-oscillatory and/or composed of many oscillatory signals at different neighboring frequencies and therefore appears non-oscillatory. In a first implementation referred to as “locally referenced power”, the “background activity” for a given frequency range of interest (here beta frequency range defined as 16-32 Hz) is estimated by interpolation from power at lower and higher neighboring frequencies, referred to herein as “reference supports” (in this example, reference support points of 12 and 40 Hz are used). Note that in cases where a peak in the alpha band is expected (e.g. a peak around 10 Hz), reference support points that are closer to the beta frequency range on the lower end of the scale may be used, such as e.g. any point between 12 and 16 Hz). The estimated “background activity” is then “removed” (i.e. excluded, such as subtracted) from the power in the frequency range of interest or a power spectral density value at a specific frequency within this frequency range (e.g. the peak frequency, or a predetermined frequency, such as e.g. a frequency identified as the group level peak in a population, e.g. 23 Hz in the present case). The resulting estimate should thereby provide a more specific measure of the oscillatory physiological process under investigation (here GABA_{A}-related beta-band oscillations).

More specifically, the “background power spectral density” at a given frequency may be derived by linear interpolation of logarithmically transformed power and frequencies (see FIG. 4B). The power signal in spectral analysis is typically log transformed by default. However, the frequency scale may not be log transformed by default and hence the method may comprise log transforming at least the frequency scale. The “background power” is then estimated as the integral of this “background power spectral density” over the beta frequency range (here defined as 16-32 Hz). In other words, the “background signal” is the area under a straight line extending between the value of the spectrum at each of the support points (“background signal line”), between 16 and 32 Hz. The “locally referenced beta power” is then defined as the logarithm of the ratio between the beta power defined as the integral over the beta frequency range and the estimate of the “background power” (which is equivalent to the logarithm of the beta power defined as the integral over the beta frequency range, minus the logarithm of the estimate of the “background power”, or the area under the power curve in log scale, in the beta frequency range, minus the area under the background signal line in log scale, in the beta frequency range). Note that this metric will be negative when there is a deficit in the signal in this range compared to background. This enables the metrics as described herein to be used to identify both excessive and defective signaling, as demonstrated further below.

In a second implementation referred to as “locally referenced beta peak power”, the power spectral density is divided by the estimated “background power spectral density” (i.e. the straight line extending between the value of the spectrum at each of the support points, at each frequency), separately for each frequency, and then the maximum value in the beta frequency range (16-32 Hz) is extracted.

In a third implementation, an approach using a global fit of the power spectrum composed of a superposition of oscillatory signals and a 1/f^{α} component was used to estimate the background signal. The 1/f^{α} component of the fit (estimated as described in Donoghue et al., 2020, see FIG. 4C—note that only step (a) of the method described in Donoghue et al. is used herein, where an aperiodic component is estimated, see FIG. 2a of Donoghue et al.) was used as the estimate of the background power spectral density. Analogously to the above, in a first embodiment of this third implementation, the extracted “1 over f referenced beta power” was used to estimate the background signal, and in a second embodiment the extracted “1 over f referenced beta peak power” was used to estimate the background signal. In other words, in a first embodiment of the third implementation, the “background power” is the area under the 1/f^{α} component of the fit, between 16 and 32 Hz, and the “locally referenced beta power” is defined as the logarithm of the ratio between the beta power defined as the integral over the beta frequency range and the estimate of the “background power”. Similarly, in a second embodiment of the third implementation, the “background power spectral density” is provided by the 1/f^{α} component of the fit, the power spectral density is divided by the estimated “background power spectral density” separately for each frequency, and then the maximum value in the beta frequency range (16-32 Hz) is extracted.

These new approaches were compared to commonly used metrics, in particular: (i) absolute beta power, i.e. EEG signal power in the beta frequency range (16-32 Hz) and (ii) relative power beta, i.e. EEG signal power in the beta frequency (16-32 Hz) range divided by to the EEG signal power across all frequencies.

Statistical Analyses. A basic characteristic of the dataset analyzed is that the participants had different numbers of visits. To account for partially redundant information from repeated visits and to not over-represent participants with more than one visit, but at the same time to use all data available, the following approach was used. All of the analyses described below were performed repeatedly on all possible permutations of the dataset with each participant contributing only one visit. The quantity of interest for the specific analysis (e.g. linear regression parameters with age, correlation coefficient) was then derived as the mean across all resamples (for correlation coefficients values were Fisher z-transformed before averaging and then back-transformed). To derive p-values and confidence intervals the number of participants was used, not the number of visits, as degrees of freedom.

Statistical comparisons of EEG metrics between groups were performed using unpaired t-test allowing unequal variance. Effect sizes were reported using Hedges' g.

To ensure that possible age-dependencies of EEG metrics and clinical scales do not lead to trivial correlations between the two, a two-step approach was used. In a first step, the effect of age was removed (linear regression of log-age) from EEG metrics and clinical scales, respectively. In a subsequent step, the data was then analysed to identify correlations between EEG metrics and clinical scales.

To analyze cross-sectional relationships between EEG metrics and clinical parameters, Pearson correlation coefficients was used (Spearman rank correlations for sensitivity analyses).The results shown below also held for non-parametric correlations (not shown). The p-values confidence intervals were derived exploiting that the Fisher z-transformed correlation coefficients are approximately normally distributed with a standard error of 1/√(n−3) (1.06/√(n−3) for Spearman rank correlations). Given our directional hypothesis (greater EEG abnormality with lower clinical scores), 1-sided tests are reported and 90% confidence intervals.

### Results

In this example, a new approach to isolate the EEG beta-band oscillations from the background signal is developed, to derive a sensitive and specific measure of excess GABA-A receptor function. It is then investigated whether this measure better separates Dup15q syndrome individuals from typical controls and whether individuals with stronger beta-band oscillations, i.e. more pronounced excess GABA-A receptor function, present with a more severe phenotype.

Inspecting the power spectra of individuals with Dup15q syndrome, the inventors recognised that a clear beta-band peak is apparent for almost all individuals (see FIG. 4A). However, the inventors also recognised that the beta-band peaks are superimposed on a background that substantially differs between individuals. This “background signal” likely reflects other neuronal processes unrelated to the excess GABA-A receptor signalling. Absolute beta power, as used in prior studies, quantifies a mixture of the beta-band oscillations and the “background signal”. Absolute beta power is therefore a rather unspecific and possibly insensitive proxy of the excess GABA-A function in Dup15q syndrome.

A frequently used approach to account for differences in overall magnitude of electrophysiological signals is to use relative power, i.e. normalizing spectral power of interest by the total power of the broad-band EEG signal. While this approach may work well for some applications, the present inventors recognised that this will likely be a poor choice for the problem at hand. Despite the prominent beta-band peaks in the Dup15q syndrome power spectra, beta power accounts for only a small fraction of the total power. Division by the total power would therefore introduce variance related to signals at lower frequencies that contain most of signal power and which, according to visual inspection, also substantially differ between individuals. Furthermore, effects found with relative beta power may arise from effects in the beta band or any other frequency range, since power at all frequencies enter the normalization, and is therefore not specific to the beta-band anymore. This affects interpretability, in particular the link to GABA-A function.

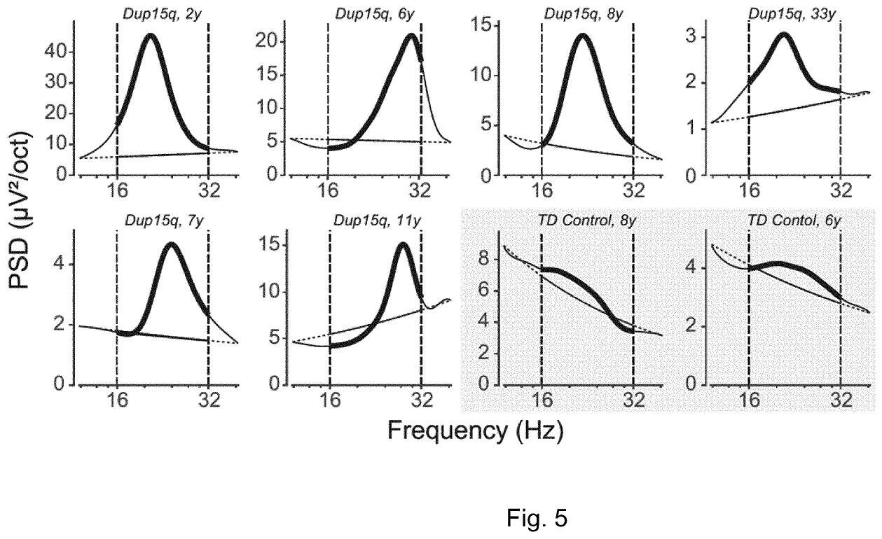

The inventors devised a new analysis approach that aims to specifically quantify the beta-band oscillations while being invariant to the background signal, i.e. “locally referenced power” (see Methods; FIG. 4B). They estimated the “background signal” in the beta-band (defined as 16-32 Hz) by interpolation from power at lower and higher neighbouring frequencies (12 and 40 Hz, respectively). They then removed the estimated “background activity” from the beta power. The extraction of “locally referenced power” is illustrated for power spectra from six individuals with Dup15q syndrome and two typical developing controls in FIG. 4D,E and FIG. 5.

The inventors also investigated variants of the approach that either differed in the estimation of the “background signal” (using the 1/f^{α} component of a global fit of the power spectrum comprising a superposition of oscillatory signals and a 1/f^{α} component; (Donoghue et al., 2020); FIG. 4C; “1 over f referenced beta power”), and/or using the maximum of the normalized power spectral density in the beta frequency range instead of the power in the beta frequency range (FIGS. 4B,C, “locally referenced beta peak power”, “1 over f referenced beta peak power”) (see Methods).

The inventors postulated that the “Locally referenced beta power”, and the variants thereof, should be a more specific quantification of oscillatory activity and should therefore be a better proxy of the excess GABAA function in Dup15q syndrome.

The inventors found that the “locally referenced beta power” was largely independent of age (Table 1; FIG. 6A) and was strongly elevated in individuals with Dup25q syndrome compared to TD controls (Table 2; locally referenced beta power, ES=1.84, FIG. 61B). The difference was spatially rather unspecific, though the effect was least pronounced at electrodes C3 and C4 (FIG. 6C). The difference between groups was much stronger compared to absolute beta power (FIG. 6F, ES=0.99, p=2.8×10⁻⁵) and relative beta power (FIG. 6G, ES: 1.21, p=2.1×10⁻⁶). The variants of “locally referenced beta power” showed very similar characteristics with even slightly stronger separation between cases and controls with an effect size of up to 2.13 (Table 2; FIGS. 6D,E).

TABLE 1
Age dependence of clinical scales and EEG beta power metrics for
individuals with Dup15q syndrome without a history of epilepsy.

| | Number of participants (recordings) | | | | | |
|---|---|---|---|---|---|---|
| | IDIC | | INT | | All Dup 15q | |
| Test/Beta power metric | All | w/EEG | All | w/EEG | All | w/EEG |
| NVDQ | 16 (22) | 13 (15) | 12 (14) | 11 (12) | 28 (36) | 24 (27) |
| VDQ | 16 (22) | 13 (15) | 12 (14) | 11 (12) | 28 (36) | 24 (27) |
| FSDQ | 16 (22) | 13 (15) | 12 (14) | 11 (12) | 28 (36) | 24 (27) |
| VABS Com | 18 (24) | 15 (17) | 12 (14) | 11 (12) | 30 (38) | 26 (29) |
| VABS DIs | 18 (24) | 15 (17) | 12 (14) | 11 (12) | 30 (38) | 26 (29) |
| VABS Soc | 18 (24) | 15 (17) | 12 (14) | 11 (12) | 30 (38) | 26 (29) |
| VABS Motor | 16 (22) | 13 (15) | 11 (13) | 10 (11) | 27 (35) | 23 (26) |
| VABS ABC | 17 (23) | 14 (16) | 11 (13) | 10 (11) | 28 (36) | 24 (27) |
| Absolute | 16 (19) | 16 (19) | 12 (14) | 12 (14) | 28 (33) | 28 (33) |
| Relative | 16 (19) | 16 (19) | 12 (14) | 12 (14) | 28 (33) | 28 (33) |
| Locally referenced | 16 (19) | 16 (19) | 12 (14) | 12 (14) | 28 (33) | 28 (33) |
| Locally referenced peak | 16 (19) | 16 (19) | 12 (14) | 12 (14) | 28 (33) | 28 (33) |
| 1 over f referenced | 16 (19) | 16 (19) | 12 (14) | 12 (14) | 28 (33) | 28 (33) |
| 1 over f referenced peak | 16 (19) | 16 (19) | 12 (14) | 12 (14) | 28 (33) | 28 (33) |
| | Correlation with log(Age) | | | | | |
| | IDIC | | INT | | All Dup 15q | |
| Test/Beta power metric | r | p | r | p | r | p |
| NVDQ | −0.29 | 0.071 | −0.45 | 0.038 | −0.24 | 0.056 |
| VDQ | −0.12 | 0.168 | −0.23 | 0.120 | −0.12 | 0.140 |
| FSDQ | −0.31 | 0.062 | −0.31 | 0.084 | −0.20 | 0.078 |
| VABS Com | −0.17 | 0.125 | 0.11 | 0.187 | 0.07 | 0.182 |
| VABS DIs | −0.61 | 0.002 | 0.11 | 0.187 | −0.23 | 0.058 |
| VABS Soc | −0.51 | 0.007 | −0.11 | 0.187 | −0.23 | 0.058 |
| VABS Motor | −0.27 | 0.080 | −0.19 | 0.145 | −0.10 | 0.159 |
| VABS ABC | −0.40 | 0.027 | 0.09 | 0.201 | −0.08 | 0.169 |
| Absolute | −0.53 | 0.008 | −0.15 | 0.163 | −0.17 | 0.072 |
| Relative | −0.23 | 0.099 | 0.48 | 0.029 | 0.10 | 0.138 |
| Locally referenced | −0.24 | 0.092 | 0.12 | 0.181 | 0.01 | 0.242 |
| Locally referenced peak | −0.22 | 0.107 | 0.10 | 0.189 | 0.03 | 0.219 |
| 1 over f referenced | −0.13 | 0.158 | 0.31 | 0.084 | 0.13 | 0.104 |
| 1 over f referenced peak | −0.12 | 0.169 | 0.25 | 0.110 | 0.11 | 0.119 |

Abbreviations. TD: typical developing controls, INT: Dup15q syndrome with interstitial duplications of 15q11-13, IDIC: isodicentric duplications, All Dup15q: Dup15q syndrome of any genotype, i.e. INT and IDIC combined, w/EEG: subject with EEG recording, r: correlation coefficient between clinical scale or EEG metric and logarithm of the age, p: p value associated with the correlation.

TABLE 2
Differences in beta power measures between
Dup15q syndrome subgroups and TD controls.

| | TD | | INT | | IDIC | |
|---|---|---|---|---|---|---|
| Beta power metric | mean | SD | mean | SD | mean | SD |
| Absolute | 6.870 | 1.210 | 8.070 | 4.220 | 9.580 | 2.800 |
| Relative | −9.520 | 1.200 | −6.970 | 1.960 | −6.960 | 2.960 |
| Locally referenced | 0.321 | 0.526 | 2.600 | 1.930 | 3.420 | 2.290 |
| Locally referenced peak | 0.998 | 0.480 | 4.070 | 2.540 | 5.200 | 2.700 |
| 1 over f referenced | 0.372 | 0.481 | 2.490 | 1.540 | 2.990 | 1.750 |
| 1 over f referenced peak | 1.160 | 0.691 | 4.030 | 2.160 | 4.840 | 2.200 |

| | All Dup15q | | INT-TD | | IDIC-TD | |
|---|---|---|---|---|---|---|
| Beta power metric | mean | SD | ES | p* | ES | p* |
| Absolute | 9.120 | 3.330 | 0.60 | 5.3E−02 | 1.25 | 9.2E−06 |
| Relative | −6.970 | 2.680 | 1.82 | 1.5E−05 | 1.05 | 9.4E−05 |
| Locally referenced | 3.170 | 2.210 | 1.98 | 1.3E−05 | 1.82 | 1.6E−09 |
| Locally referenced peak | 4.850 | 2.680 | 2.00 | 1.6E−05 | 2.12 | 3.8E−11 |
| 1 over f referenced | 2.840 | 1.690 | 2.07 | 7.7E−06 | 2.00 | 1.4E−10 |
| 1 over f referenced peak | 4.590 | 2.200 | 1.94 | 1.6E−05 | 2.21 | 9.7E−12 |

| | DUP15q-TD | | IDIC-INT | | IDIC:Epi:NoEpi | |
|---|---|---|---|---|---|---|
| Beta power metric | ES | p | ES | p | ES | p |
| Absolute | 0.99 | 2.8E−05 | 0.29 | 1.7E−01 | −0.57 | 1.1E−01 |
| Relative | 1.21 | 2.1E−06 | −0.13 | 6.7E−01 | −0.47 | 1.8E−01 |
| Locally referenced | 1.84 | 7.4E−13 | 0.25 | 2.1E−01 | −0.33 | 3.5E−01 |
| Locally referenced peak | 2.07 | 5.6E−15 | 0.31 | 1.6E−01 | −0.28 | 4.2E−01 |
| 1 over f referenced | 2.02 | 5.6E−14 | 0.20 | 2.6E−01 | −0.25 | 4.7E−01 |
| 1 over f referenced peak | 2.13 | 1.6E−14 | 0.27 | 1.9E−01 | −0.18 | 6.1E−01 |

Abbreviations. TD: typical developing controls, INT: Dup15q syndrome with interstitial duplications of 15q11-13, IDIC: isodicentric duplications, Dup15q: Dup15q syndrome of any genotype, i.e. INT and IDIC combined, Epi: Individuals with epilepsy. NoEpi: Individuals without epilepsy, SD: standard deviation, ES: effect size, p: p value from an unpaired t-test with unequal variance between groups indicated in the title line.
*1-tailed test.

Locally relative beta power was numerically higher in IDIC (triplication of 15q11-13) compared to INT (duplication of 15q11-13), i.e. the expected direction, but the distributions are largely overlapping and differences were not significant (Table 2; ES=0.25, p=0.21; FIG. 6B). A topographic analysis of the difference suggested that there may be higher locally relative beta power in IDIC compared to INT at temporal electrodes but this needs further investigation with more data in future studies (FIG. 6C). Similarly, the diagnosis of epilepsy did not have a significant impact on the magnitude of beta power (FIG. 6B).

The substantially better separation of individuals with Dup15q syndrome and TD (typically developing) controls of “locally referenced beta power” (and variants thereof) compared to absolute power used in previous studies, indeed suggests that it better captured beta oscillations in Dup15q syndrome. Furthermore, there was substantial spread in “locally referenced beta power” across the Dup15q syndrome population and a high test-retest reliability. Together these characteristics strongly support investigating the relationship with symptom severity.

About half of the individuals with Dup15q syndrome develop epilepsy. In prior work the inventors found that individuals with Dup15q syndrome and epilepsy were much more severely impaired than individuals without epilepsy (DiStefano et al., 2020)). This is likely a reflection of epilepsy further disrupting neuronal development beyond more specific contribution of the copy number gains of genes on 15q11-13. To investigate the principled question of a contribution of the GABR subunit gene cluster to Dup15q syndrome the inventors opted to test for a relationship between EEG and symptom severity in individuals without epilepsy. This should reduce variance in clinical severity related to, possibly unspecific, effects of epilepsy and reduce problems related to flooring effects of the clinical scales in the more impaired part of the population observed in the inventor's prior work. Note that epilepsy has no impact on use of the metrics described herein for the uses described herein (i.e. for any other use than the particular example of predicting symptom severity in individuals with Dup15q syndrome).

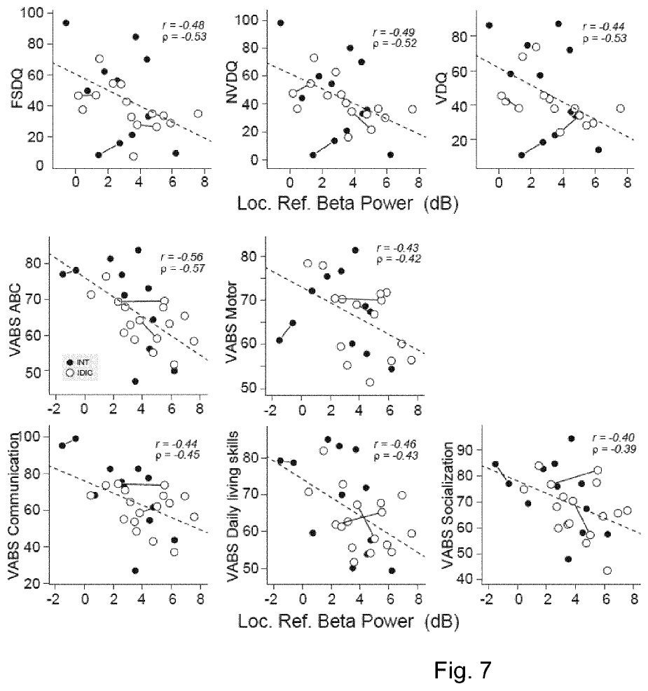

The correlation of “locally referenced beta power” and all three variants thereof were significant for all scales investigated (p<0.05, r: −0.56-−0.40, Table 4, FIG. 7). The negative sign of the correlation is in the expected direction, i.e. higher EEG beta-band oscillations go in hand with a more severe symptoms (as quantified using the FSDQ (full scale DQ) and VABS ABC summary measures). The variants of “locally referenced beta power” showed very similar characteristics with even slightly stronger correlations of up to r=−0.59 (Table 4). In line with previous work (Saravanapandian et al., 2020), the correlation was not found with absolute beta power and only for few scales and much weaker for relative beta power (Table 3).

The correlation analyses described herein accounted for possible effects of age by regressing out age effects from both the clinical scales and the locally referenced beta power before performing the correlation. As a sensitivity analysis the inventors performed the same analysis without this age correction and found highly similar results.

After finding significant correlations in individuals with Dup15q syndrome without epilepsy the inventors asked of this correlation could also be seen in individuals with epilepsy despite the problems related to additional epilepsy related variance and flooring effects. The correlation analysis in individuals with Dup15q syndrome and epilepsy did not reveal significant correlations (p>0.05 for all scales).

TABLE 3
Correlation between locally referenced beta power
and clinical severity in Dup15q syndrome.

| Beta | | | | | | | | | | |
|---|---|---|---|---|---|---|---|---|---|---|
| power | Clinical | | | | | | | rho | rho | r |
| metric | Scale | r | p | r LB | r UB | rho | p | LB | UB | Average |
| Absolute | NVDQ | −0.35 | 0.047 | −0.62 | −0.01 | −0.43 | 0.021 | −0.68 | −0.09 | −0.19 |
| | VDQ | −0.28 | 0.097 | −0.57 | 0.08 | −0.34 | 0.056 | −0.62 | 0.01 | |
| | FSDQ | −0.32 | 0.066 | −0.60 | 0.03 | −0.38 | 0.040 | −0.64 | −0.03 | |
| | VABS | −0.22 | 0.146 | −0.51 | 0.12 | −0.20 | 0.174 | −0.50 | 0.15 | |
| | Com | | | | | | | | | |
| | VABS | −0.27 | 0.090 | −0.55 | 0.06 | −0.30 | 0.074 | −0.58 | 0.04 | |
| | DIs | | | | | | | | | |
| | VABS | −0.21 | 0.154 | −0.51 | 0.13 | −0.19 | 0.190 | −0.49 | 0.16 | |
| | Soc | | | | | | | | | |
| | VABS | 0.24 | 0.859 | −0.13 | 0.54 | 0.20 | 0.809 | −0.18 | 0.52 | |
| | Motor | | | | | | | | | |
| | VABS | −0.11 | 0.307 | −0.44 | 0.24 | −0.15 | 0.254 | −0.48 | 0.22 | |
| | ABC | | | | | | | | | |
| Relative | NVDQ | −0.42 | 0.020 | −0.67 | −0.09 | −0.33 | 0.066 | −0.61 | 0.03 | −0.35 |
| | VDQ | −0.38 | 0.035 | −0.64 | −0.04 | −0.36 | 0.047 | −0.63 | −0.01 | |
| | FSDQ | −0.42 | 0.020 | −0.67 | −0.09 | −0.37 | 0.043 | −0.64 | −0.02 | |
| | VABS | −0.29 | 0.076 | −0.57 | 0.04 | −0.26 | 0.112 | −0.55 | 0.09 | |
| | Com | | | | | | | | | |
| | VABS | −0.39 | 0.025 | −0.64 | −0.07 | −0.29 | 0.086 | −0.57 | 0.06 | |
| | DIs | | | | | | | | | |
| | VABS | −0.27 | 0.095 | −0.55 | 0.07 | −0.15 | 0.235 | −0.47 | 0.20 | |
| | Soc | | | | | | | | | |
| | VABS | −0.24 | 0.142 | −0.54 | 0.13 | −0.20 | 0.192 | −0.52 | 0.18 | |
| | Motor | | | | | | | | | |
| | VABS | −0.39 | 0.031 | −0.64 | −0.05 | −0.31 | 0.075 | −0.60 | 0.05 | |
| | ABC | | | | | | | | | |
| Locally | NVDQ | −0.49 | 0.007 | −0.72 | −0.18 | −0.52 | 0.006 | −0.74 | −0.20 | −0.46 |
| referenced | VDQ | −0.44 | 0.015 | −0.68 | −0.12 | −0.53 | 0.004 | −0.74 | −0.22 | |
| | FSDQ | −0.48 | 0.008 | −0.71 | −0.16 | −0.53 | 0.004 | −0.74 | −0.22 | |
| | VABS | −0.44 | 0.012 | −0.67 | −0.13 | −0.45 | 0.013 | −0.68 | −0.13 | |
| | Com | | | | | | | | | |
| | VABS | −0.46 | 0.009 | −0.68 | −0.15 | −0.44 | 0.015 | −0.68 | −0.11 | |
| | DIs | | | | | | | | | |
| | VABS | −0.40 | 0.020 | −0.65 | −0.08 | −0.40 | 0.026 | −0.65 | −0.06 | |
| | Soc | | | | | | | | | |
| | VABS | −0.43 | 0.020 | −0.68 | −0.09 | −0.42 | 0.026 | −0.68 | −0.07 | |
| | Motor | | | | | | | | | |
| | VABS | −0.56 | 0.002 | −0.76 | −0.27 | −0.57 | 0.002 | −0.77 | −0.27 | |
| | ABC | | | | | | | | | |
| Locally | NVDQ | −0.52 | 0.004 | −0.74 | −0.22 | −0.54 | 0.004 | −0.75 | −0.23 | −0.49 |
| referenced | VDQ | −0.48 | 0.008 | −0.71 | −0.16 | −0.54 | 0.004 | −0.75 | −0.23 | |
| peak | FSDQ | −0.51 | 0.005 | −0.73 | −0.21 | −0.55 | 0.003 | −0.75 | −0.24 | |
| | VABS | −0.45 | 0.010 | −0.68 | −0.14 | −0.47 | 0.010 | −0.69 | −0.15 | |
| | Com | | | | | | | | | |
| | VABS | −0.52 | 0.003 | −0.73 | −0.23 | −0.50 | 0.006 | −0.72 | −0.19 | |
| | DIs | | | | | | | | | |
| | VABS | −0.43 | 0.014 | −0.67 | −0.12 | −0.44 | 0.015 | −0.68 | −0.11 | |
| | Soc | | | | | | | | | |
| | VABS | −0.45 | 0.015 | −0.69 | −0.12 | −0.43 | 0.024 | −0.68 | −0.08 | |
| | Motor | | | | | | | | | |
| | VABS | −0.58 | 0.001 | −0.77 | −0.29 | −0.57 | 0.002 | −0.77 | −0.27 | |
| | ABC | | | | | | | | | |
| 1 over f | NVDQ | −0.51 | 0.005 | −0.73 | −0.20 | 0.49 | 0.008 | −0.72 | −0.17 | −0.49 |
| referenced | VDQ | −0.46 | 0.011 | −0.70 | −0.14 | −0.51 | 0.006 | −0.73 | −0.19 | |
| | FSDQ | −0.49 | 0.007 | −0.72 | −0.18 | −0.51 | 0.006 | −0.73 | −0.19 | |
| | VABS | −0.42 | 0.015 | −0.66 | −0.11 | −0.45 | 0.013 | −0.68 | −0.12 | |
| | Com | | | | | | | | | |
| | VABS | −0.50 | 0.004 | −0.72 | −0.21 | −0.47 | 0.009 | −0.70 | −0.15 | |
| | DIs | | | | | | | | | |
| | VABS | −0.45 | 0.011 | −0.68 | −0.14 | 0.46 | 0.010 | −0.69 | −0.14 | |
| | Soc | | | | | | | | | |
| | VABS | −0.47 | 0.012 | −0.70 | −0.14 | −0.43 | 0.023 | −0.69 | −0.08 | |
| | Motor | | | | | | | | | |
| | VABS | −0.58 | 0.001 | −0.77 | −0.30 | −0.58 | 0.002 | −0.77 | −0.28 | |
| | ABC | | | | | | | | | |
| 1 over f | NVDQ | −0.53 | 0.003 | −0.74 | −0.23 | −0.52 | 0.005 | −0.74 | −0.21 | −0.51 |
| referenced | VDQ | −0.48 | 0.008 | −0.71 | −0.17 | −0.52 | 0.005 | −0.74 | −0.20 | |
| peak | FSDQ | −0.52 | 0.004 | −0.73 | −0.21 | −0.53 | 0.004 | −0.75 | −0.22 | |
| | VABS | −0.42 | 0.016 | −0.66 | −0.11 | −0.45 | 0.013 | −0.68 | −0.13 | |
| | Com | | | | | | | | | |
| | VABS | −0.56 | 0.001 | −0.75 | −0.28 | −0.51 | 0.004 | −0.73 | −0.21 | |
| | DIs | | | | | | | | | |
| | VABS | −0.45 | 0.010 | −0.68 | −0.14 | 0.45 | 0.012 | −0.69 | −0.13 | |
| | Soc | | | | | | | | | |
| | VABS | −0.48 | 0.010 | −0.71 | −0.15 | −0.42 | 0.027 | −0.68 | −0.06 | |
| | Motor | | | | | | | | | |
| | VABS | −0.59 | 0.001 | −0.78 | −0.30 | −0.58 | 0.002 | −0.77 | −0.28 | |
| | ABC | | | | | | | | | |

Abbreviations,
LB: Lower bound,
UB: Upper bound

### Discussion

Using a new analysis approach, the present inventors isolated beta-band oscillations from the EEG of individuals with Dup15q syndrome as a biomarker of excess GABA-A receptor function. They found a correlation of the strength of these oscillations with symptom severity across all symptom domains investigated. The results provide novel evidence that excess GABA-A receptor signaling is contributing to the etiology of Dup15q syndrome, as well as a new, more sensitive approach to detect GABA-A receptor dysfunction. Importantly, this work provides evidence for the utility and sensitivity of the procedure subject to the patent to quantify excess GABA-A function.

The GABA-A receptor gene cluster on 15q11-13 encodes the α5, β3, and γ3 GABA-A receptor subunits, which (along with γ2 GABA-A receptor subunit) co-assemble to form functional GABA-A α 5-containing receptor subtype. Thus, the excess GABA-A function is Dup15q is likely caused by an overabundance of GABA-A α5Rs suggesting this receptor subtype as a therapeutic target for Dup15q syndrome. Antagonists or preferably negative allosteric modulators (NAM, given their superior safety profile) that are selective to GABA-A α5R, like basmisanil (Hipp et al., 2021), may therefore constitute new treatment options. This is supported by the EEG effect of the GABA-A α5 NAM basmisanil, which has been investigated in healthy volunteers and individuals with down-syndrome, and is characterized by a decrease in beta-band power (Goeldner et al., 2022; Hipp et al., 2021).

The present work suggests that “locally referenced beta power” and variants thereof are a useful cross-sectional biomarker of the excess GABA-A receptor related pathophysiology in Dup15q syndrome. These metrics' utility is demonstrated by the high separation compared to controls and the correlation with symptom severity. The different variants showed very similar results and they may be used interchangeably in the present context. In a particular context or application, one or more of the metrics described herein may be preferred if they result in beneficial effects such as e.g. increased sensitivity. The skilled person will be able to assess which one or more of the metrics described herein provides a particular benefit (e.g. sensitivity) in relation to making a particular determination.

Individuals with a history of epilepsy are substantially more impaired (DiStefano et al., 2020)), which likely reflects additional pathophysiology related to the epilepsy and also goes in hand with the problem of the clinical scales used showing flooring effects, i.e. not adequately capturing the symptoms. The inventors believe that it is plausible that a correlation with symptom severity was not found in the sub-group with epilepsy for those two reasons, i.e. strong limitations to capture the symptoms and additional pathophysiology that contributes to the severity. As such, explaining the epilepsy-related variance (e.g. by some metrics quantifying epilepsy severity and duration) and/or using clinical scales that better capture symptoms in this population, should allow finding the correlation also in the sub-population with epilepsy. In view of this, the present results in the non-epileptic sub-population provide evidence for a role of excess GABA-A receptor signaling in the pathophysiology if Dup15q syndrome.

The present results provide evidence for the relevance of GABA-A receptors in addition to UBE3A. The differential contribution of the GABA-A receptors and UBE3A remains an open question. It should be noted that their contribution might also be highly non-additive, i.e. dependent on each other. E.g. it is conceivable that addressing either one of the problem had a dramatic effect, while alternatively, it may be the case that both need to be addressed jointly to have a significant effect.

The resting state or background EEG signal (and related electrophysiological signals, e.g. MEG) is a superposition of various oscillatory and non-oscillatory signals reflecting many different neuronal processes. It is highly characteristic for an individual. The power spectrum is a useful frequency domain representation of the EEG. Oscillatory activity, like e.g. the beta-band oscillations of interest in this work, resides at specific frequencies in the power spectrum but can be “confounded” by “background activity”, e.g. non-oscillatory processes. “Locally referenced power” and the related metrics provide an analysis approach to isolate oscillatory signals at specific frequencies from the background activity and should therefore be more specific and sensitive representation of oscillatory signals of interest. The present finding that effect sizes between groups (Dup15q vs. neurotypical controls) are substantially larger and that the correlation with symptom severity only emerge when using “locally relative power”, compared to absolute power, indeed supports the utility of the approach. Notably, “locally referenced power” may not only be used to identify excess oscillatory signals, i.e. peaks in the power spectrum, but also reduced oscillatory signals, i.e. troughs in the power spectrum (as demonstrated further below, which may be thought of as lower than normal oscillatory activity for a given oscillatory process).

Relative power, which is a frequently used metric normalizing the power spectrum by the overall power, showed higher effect sizes in group comparisons compared to absolute power, and correlation with a few clinical scales. However, the effects were weaker compared to “locally relative power”. Importantly, relative power suffers from the problem of interpreting the results since signal power at the frequency of interest is influenced by power at all other frequencies. “Locally referenced power” does not suffer from the problem of depending on power at all other frequencies since the normalization is derived from interpolation using only the signal from frequencies directly neighboring the frequency of interest, i.e. “local” signals. In turn, however, “locally referenced power” depends on the choice of the “support points”, i.e. the points for interpolation. The choice of these “support points” needs to be chosen carefully for the application to not tap into other salient oscillations in the power spectrum. E.g. the choice of 12 Hz for one of the “support points” used in this study may be problematic in the presence of strong alpha peaks as found in typical developing individuals. In neurodevelopmental disorders like Dup15q syndrome, alpha activity is substantially reduced in amplitude or shifted to lower frequencies and 12 Hz was therefore considered a good “support point”. Notably, in the context of a treatment, changes occurring at the “support points” could confound the results (e.g. emergence of an alpha peak when studying “locally referenced beta power”. Thus, the choice of suitable support point may be made by examination of the data to be analysed, preferably to select points that are not influenced by a confounding effect (e.g. effect of treatment) and/or that do not show relevant activity (e.g. alpha peaks, activity in response to treatment, etc.). The skilled person would be able to determine points that are suitable for this based on one or more power spectra to be analysed.

The inventors explored a variant of the approach, i.e. “1 over f referenced beta power”, that derives the estimate of the background activity by using the 1/f^{α} component of a fit consisting of a superposition a 1/f^{α} component and oscillatory signals. This variant does not require to define “support points” and shows very similar, numerically even higher, separation between groups and correlation with symptom severity. However, conceptually, the approach does not allow for reduced oscillatory signals, i.e. troughs in the power spectrum as seen e.g. in Angelman syndrome (Frohlich et al., 2019b), but may still be of utility in such cases (see also Example 2). The present inventors explored two means to extract the actual beta-power values, i.e. integral over a range (16-32 Hz) and the peak of the “locally” or “1 over f” normalized power spectrum. Results were comparable but the peak-based metrics showed numerically larger group effect sizes and correlations. Notably, the peak-based metric is not readily applicable to decreases in oscillatory activity as e.g. expected for Angelman syndrome where a minimum would be more appropriate (see Example 2).

Overall, the different variants explored behave similar and support that the observed correlation is robust to details of the locally normalized approach. These results provide evidence for the sensitive and utility of the methods described herein to quantify reduced GABA-A function. The approach may be extended to the use of a data driven approach comprising learning a kernel that best separates one or more groups and/or maximises the correlation with one or more severity metrics based on the power spectrum in the beta band and neighboring frequencies. For example, the method may comprise obtaining an estimate of the power in the frequency range of interest (e.g. beta band) and an estimate of the power in said frequency range using a respective plurality of points in the frequency range and within a distance from the boundary of said frequency range (i.e. neighboring frequencies), wherein the power at said points is weighted using respective weights that are learned so as to best separate subjects with a known neurological dysfunction (e.g. subjects with Dup15q, AS, etc. —optionally distinguishing two groups based on the direction of the dysfunction) from typically developing subjects/healthy controls, or to obtain the strongest correlation between the resulting metric and a symptom metric of interest.

### Example 2—Oscillatory EEG Activity in the Beta-Frequency Range Indicates Insufficient GABA-A Receptor Function in Angelman Syndrome

In this example, the inventors used the approach developed in Example 1 to analyse data from patients with Angelman syndrome. Deletion Angelman syndrome is with respect to three GABA-A genes the converse and should accordingly go in hand with reduced locally referenced beta power and separate from typical developing controls.

### Methods

See Example 1.

Datasets. Data from the FREESIAS dataset was used. FREESIAS is a prospective, observational, longitudinal study conducted in individuals with AS and typically developing controls (TDC) to characterize the clinical features of individuals with AS and TDC over a period of 12 months.

The study was carried out at six sites in the United States (Boston Children's Hospital, Boston, Massachusetts; Rady Children's Hospital, San Diego, California; Rush University Medical Center, Chicago, Illinois; Baylor College of Medicine, Houston, Texas; University of California, Los Angeles, California; University of North Carolina, Chapel Hill, North Carolina) between September 2019 and May 2021 (last study visit). Eligible participants were children (1-12 years of age) and adults (≥18 years of age) with AS, and TDC children aged 1-12 years. Key inclusion criteria included a detailed molecular diagnosis of AS, and caregivers being willing to provide written informed consent, comply with study requirements and accompany participants to clinic visits. Key exclusion criteria included having an unrelated medical condition that might significantly interfere with AS assessment, and current, planned (within the study duration) or previous participation (within 4 weeks) in an investigational drug or device trial. Here only data from TDC and AS with deletion genotype (see above) are shown.

Overnight EEG/limited PSG (polysomnography) recordings were performed in the participants' home. To this end, an EEG tech visited the home in the afternoon, mounted the EEG electrodes and other sensors, ensured good quality recording, instructed caregivers about the overnight assessment and then left the home to say in a hotel nearby to be able to return in case of emerging technical problems (e.g. electrodes are pulled of). Participants were then going to their beds continuing wearing the limited PSG equipment. Upon awaking the next morning, the EEG tech was informed to return to the home, additional awake EEG was recorded and then the equipment was removed. The EEG tech identified and documented 10 min of awake EEG data in the afternoon of the first day and the morning of the second day for the use of quantitative analyses.

Data were recorded with Tracklt™ Mk3 (Lifelines Neuro, sampling rate 400 Hz for electrophysiological signals) and comprised the following sensors: 19 EEG channels (10/20 montage, reference: FC5), 1 ECG channel referenced to the EEG reference. Two EOG channels (LUE, RAE), 2 leg electromyography (EMG; left leg, right leg), 1 abdominal belt, 1 chin EMG, 1 pulse Oximeter (was discontinued after a few initial recordings). We refer to this setup as “limited PSG” to acknowledge that not all sensors of a PSG were deployed. Furthermore, the participant was monitored with an infrared camera during sleep.

For this data, 10 min of awake EEG data recorded on the first day of the first home visit were analyzed quantitatively following the procedures described in Example 1.

### Results

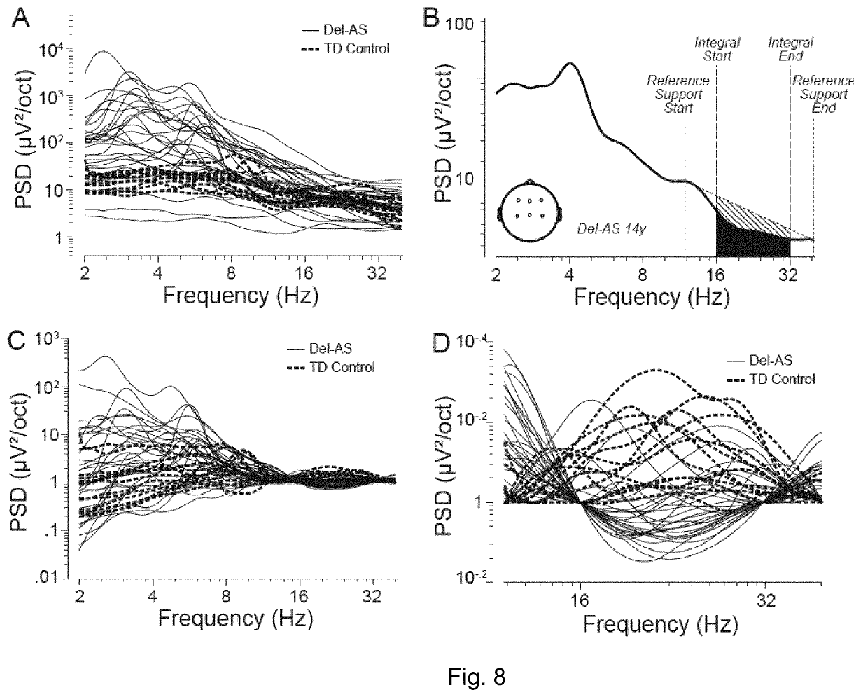

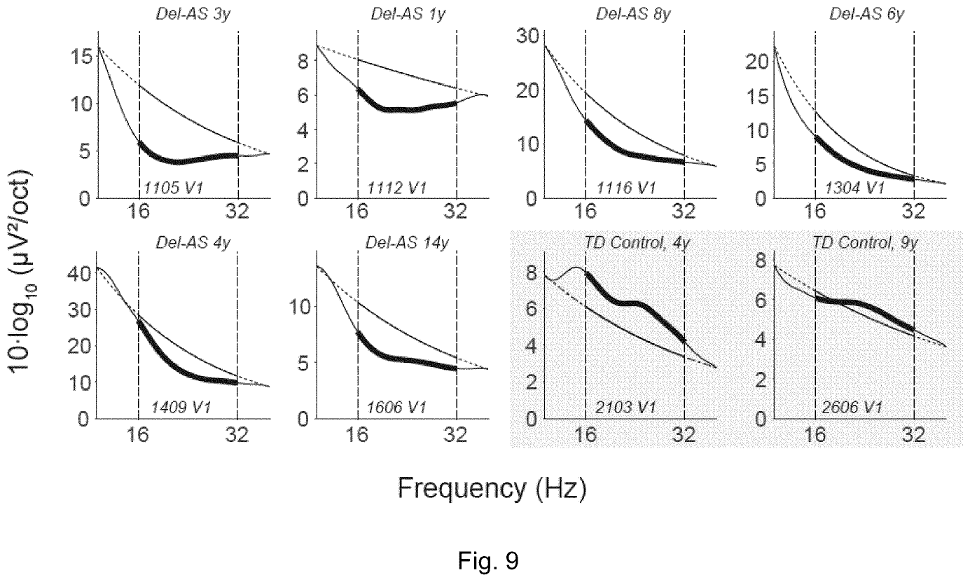

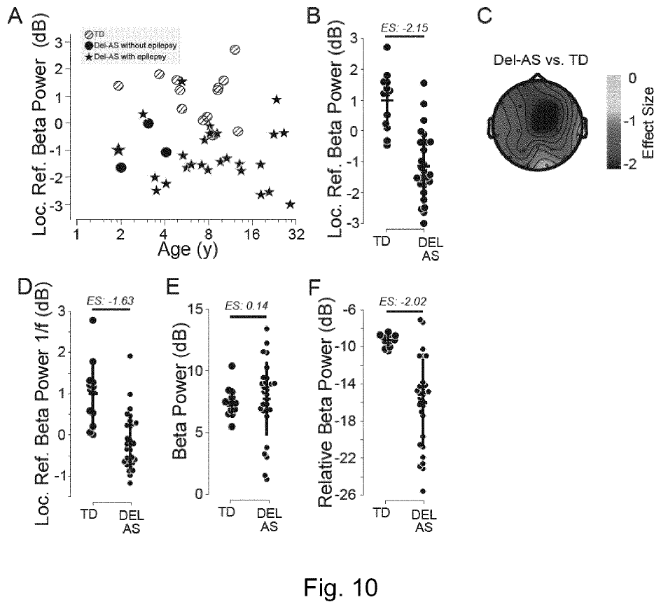

FIGS. 8 and 9 show, that when applying the procedure described in Example 1 to derive a locally referenced beta power, a decrease can be identified, indicating a deficit in GABA-A function subjects with AS. In particular, it can be seen how beta band of power drops below the estimated background signal for individuals with AS but not for TDCs (FIGS. 8C,D). “Locally referenced beta power” is substantially lower in deletion AS compared to TDCs largely independent of age (FIG. 10A,B) indicating reduced GABA-A function as expected for deletion AS (see section on Angelman syndrome above). This finding is particularly relevant, since absolute power does not reveal this difference. Absolute beta power is even higher for deletion AS compared to controls (FIG. 10E), which relates to the overall background activity being higher (see Frohlich et al., 2019a). Relative beta power shows decreased beta power (FIG. 10F) but with a smaller effect size. Notably relative beta power is barely interpretable given the pronounced phenotype of excess power at low frequencies, which enters the normalization. In particular, relative beta power will likely reflect excess delta power rather than decreased beta power. The variant of “locally referenced beta power” using a 1/f fit (described in FIG. 4C) also shows a separation but of lower magnitude (FIG. 10D). The lower performance in separating groups compared to the interpolation approach (described in FIG. 4B) may relate to the 1/f approach conceptually not allowing for reduced oscillatory signals, i.e. troughs in the power spectrum.

### Discussion

These results provide evidence for the utility and sensitivity of the methods described herein to quantify reduced GABA-A function.

### Example 3—Oscillatory EEG Activity as an Indicator of GABA-A Receptor Function Modulation Sensitivity in Autism Spectrum Disorders

In this example, the inventors built on the results in Examples 1 and 2 to show that the beta band power metrics described herein can be used to monitor the effects of GABA-A receptor function modulation, and to show how this can be used to determine GABA-A receptor function modulation sensitivity in Autism Spectrum Disorders.

### Methods

EEG dataset. Resting state EEG (eyes closed and eyes open) was recorded at 8 pre-defined timepoints per recording day for approximately 10 minutes with a 21 channel EEG system (Refa-40, Twente Medical Systems International [TMSi]B.V., the Netherlands) within the Ph1 SAD/MAD study BP40091 (clinicaltrial.gov identifier: NCT02083380). Thus, study investigated alogabat, a GABA-A α5 receptor selective positive allosteric modulator at 10 different doses and placebo. The study investigated adult healthy volunteers. Here only data from the SAD and the first day of the MAD with high quality data at pre-dose baseline and about 180 min after dosing are reported (for n see FIG. 11).

For analysis of the EEG data see Examples 1-2.

### Results and Discussion

As explained above, common disorders (e.g. ASD) are likely a mixture of individuals with different pathophysiologies, in particular a subgroup with excess GABA-A function and a subgroup with diminished GABA-A function. Indeed, both diseases with too much GABA-A function (e.g. Dup15q syndrome) or too little GABA-A function (e.g. Angelman syndrome) have significant symptom overlap with ASD, and a proportion of patients with these diseases would be classified as having ASD in the absence of a more precise genetic diagnosis.

Emerging data from genetic neurodevelopmental disorders (Dup15q syndrome, Angelman syndrome) and intervention with GABA-A receptor activity modulating drugs (Incl. Benzodiazepines) suggest that EEG beta-band power reflects both excess and diminished GABA-A receptor activity. This is further exemplified in Examples 1 and 2, which further provide an improved method to detect GABA-A dysfunction from EEG data.

### Effect of a GABA-A α5 PAM

The GABA-A α5 PAM alogabat increases EEG beta-band power relative to pre-dose baseline as compared to placebo treated controls (FIG. 11A). This pharmacodynamic effect is indeed specific to the beta-band (FIG. 11B). In line with this finding, a GABA-A α5 NAM (basmisanil from Roche) was shown to decrease EEG beta-band power (Hipp et al., 2021; see FIG. 6 of the paper). While a change from baseline analysis is optimal for identifying pharmacodynamics effect, the inventors tested the performance of the “locally referenced power” described in example 1 in a parallel group design. In particular, the inventors compared participants with alogabat and controls without taking the pre-dose baseline into account. Individuals with alogabat mimic in this case individuals with excess GABA-A function. Absolute beta power is not able to discriminate between the alogabat and the placebo group (FIG. 11A), while the two variants of “locally referenced beta power” (described in FIGS. 4B,C) allow a significant separation (effect size ˜0.7, p<0.007). Relative power does allow discriminating the groups as well, though with a lower effect size (ES: 0.62, p=0.02) and see example 1 for a discussion of problems with interpreting relative power.

The pharmacodynamic effect of alogabat simulates a condition with excess GABA-A α5 receptor function. The results show that using the procedure subject to the patent allows quantifying this effect. This provides evidence for the utility and sensitivity to GABA-A receptor modulation of the measure described in the patent.

### Utility in ASD

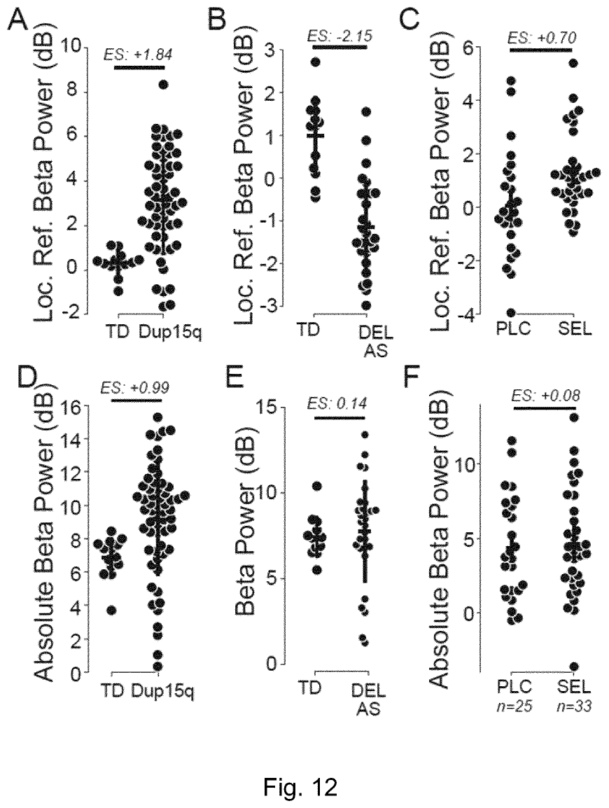

FIG. 12 summarises the results above and shows that the “locally referenced beta power” described in Example 1 separates individuals with syndromic neurodevelopmental disorders that either have excess GABA-A receptor function (example 1, Dup15q syndrome) or decreased GABA-A receptor function (example 2, Angelman syndrome) from typical developing controls and to distinguish typical developing individuals that received a GABA-A a5 PAM (alogabat) from placebo controls (example 3). Previously known methods for quantifying power in a particular section of the spectrum, in this case the beta band, i.e. using the absolute beta power, performs less well or is not capable of revealing these findings. As a result, prior art methods for identifying a difference in the beta-band power between subjects with NDD and typically developing subjects would fail to identify the correct effect in this region for many subjects and would not be of much utility. This would lead to subjects being misidentified as not having a deficiency or excess GABA-A signalling, and hence for many subjects would fail to identify a therapy that the subjects would benefit from.

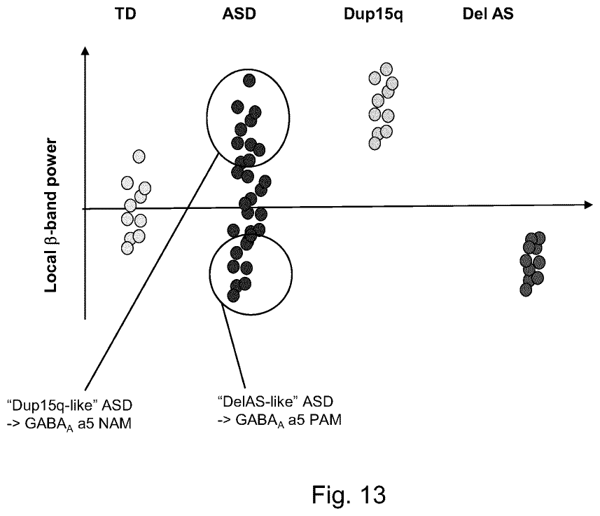

FIG. 13 illustrates schematically how the insights described herein (presence of an EEG beta-Band Signature) can be used in idiopathic ASD. In particular, the metrics described herein can be quantified for individuals diagnosed as having an autism spectrum disorder The insights provided herein suggest that some individuals with ASD may have increased “locally referenced beta power”, similar to Dup15q syndrome, indicating excess GABA-A function and may benefit from GABA-A negative modulation (e.g. basmisanil), while others individuals with ASD may have decreased beta power, similar to Angelman syndrome, indicating reduced GABA-A function and may benefit from GABA-A positive modulation (e.g. alogabat). Thus, the present inventors have identified that by quantifying the EEG beta-band signature (presence of a higher or lower locally background corrected beta-band power signal compared to TD) in ASD individuals, it would be possible to identify a subgroup of ASD individuals that as “AS-like” or have deficient GABA-A function, and/or a subgroup of individuals that are “Dup15q-like” or have excessive GABA-A function. Similarly it would therefore also be possible to identify a subgroup of ASD individuals that may benefit from treatment with a GABA-A positive modulator, and/or a subgroup of individuals that may benefit from treatment with a GABA-A negative modulator. As further explained herein, it would also be possible to identify a subgroup of ASD individuals that may suffer negative effects from treatment with a GABA-A positive modulator, and/or a subgroup of individuals that may suffer negative effects from treatment with a GABA-A negative modulator. Similarly, it would be possible to quantify the likely extent to which a subject may benefit from treatment with a GABA-A positive modulator or a GABA-A negative modulator.

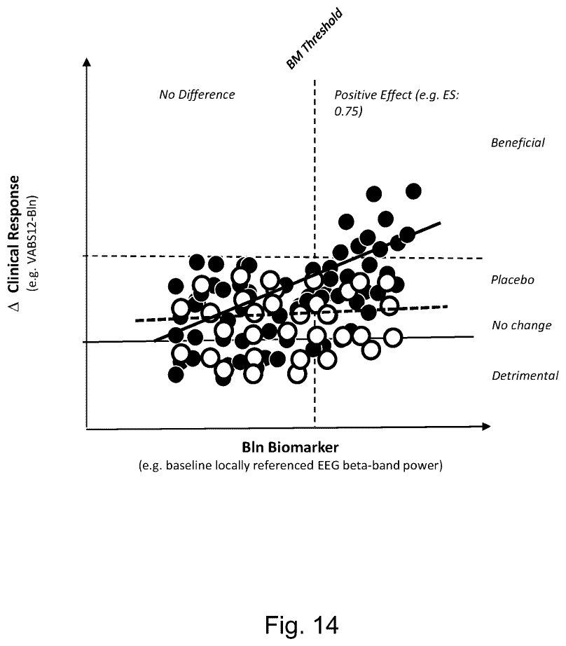

Further, the present inventors have also recognised that the metrics described herein can be used to identify a subgroup of subjects with ASD that respond to a modulator of GABA-A. For example, FIG. 14 illustrates this process for responders to a GABA-A α5 PAM. In particular, a metric as described herein (e.g. locally referenced EEG beta-band power) may be quantified at baseline (i.e. prior to treatment, x-axis), and following treatment with a GABA-A a5 PAM (Δclinical response, y-axis). A subgroup of individuals that were deficient in GABA-A function would likely benefit from the treatment and show an increase in the metric following treatment. A further subgroup of individuals that had no GABA-A dysfunction would likely not benefit from the treatment and may not show any increase in the metric following treatment. A further subgroup of individuals that had GABA-A excessive function would likely see adverse effects from the treatment and may not show any increase in the metric following treatment. Thus, the present method can be used to identify subgroups of individuals that show a beneficial effect (above placebo Δclinical response increase).

### REFERENCES

Absalom et al. (2020). Gain-of-function GABRB3 variants identified in vigabatrin-hypersensitive epileptic encephalopathies. Brain Commun. 2.
Akshoomoff (2006). Use of the Mullen Scales of Early Learning for the assessment of young children with Autism Spectrum Disorders. Child Neuropsychol. 2006 August; 12(4-5):269-77.
American Psychiatric Association, Diagnostic and Statistical Manual of Mental Disorders (5th ed.) (American Psychiatric Association Publishing, 2013).
Bird L M (2014): Angelman syndrome: Review of clinical and molecular aspects. Appl Clin Genet 7:93.
Bishop, Guthrie, Coffing, & Lord, 2011. Convergent validity of the Mullen Scales of Early Learning and the differential ability scales in children with autism spectrum disorders. Am J Intellect Dev Disabil. 2011 September; 116(5):331-43.
Bruining et al., 2020. Scientific Reports volume 10, Article number: 9195 (2020).
Chambers et al. (2003). Identification of a novel, selective GABAA α5 receptor inverse agonist which enhances cognition. J. Med. Chem. 2003; 46:2227-2240.
Chao et al., 2010. Nature volume 468, pages263-269 (2010)
Contractor,Ethell & Portera-Cailliau, 2021. Nat Neurosci. 2021 December; 24(12):1648-1659.
DiStefano et al. (2020). Behavioral characterization of dup15q syndrome: Toward meaningful endpoints for clinical trials. Am. J. Med. Genet. A. 182, 71-84.
DiStefano et al. (2016). Identification of a distinct developmental and behavioral profile in children with Dup15q syndrome. J. Neurodev. Disord. 8.
Donoghue et al. (2020). Parameterizing neural power spectra into periodic and aperiodic components. Nat. Neurosci. 23, 1655-1665.
Farmer, Golden, & Thurm, 2016. Concurrent validity of the differential ability scales, second edition with the Mullen Scales of Early Learning in young children with and without neurodevelopmental disorders. Child Neuropsychol. 2016; 22(5):556-69.
Friedman, H. et al. Pharmacokinetics and pharmacodynamics of oral diazepam: Effect of dose, plasma concentration, and time. Clin. Pharmacol. Ther. 52, 139-150.
Frohlich et al. (2016). A Quantitative Electrophysiological Biomarker of Duplication 15q11.2-q13.1 Syndrome. PLOS ONE 11, e0167179.
Frohlich et al. (2019a). Mechanisms underlying the EEG biomarker in Dup15q syndrome. Mol. Autism 10, 29.
Frohlich, et al. (2019b). Electrophysiological phenotype in Angelman syndrome differs between genotypes. Biol. Psychiatry.
Goeldner et al., 2022. Journal of Neurodevelopmental Disorders volume 14, Article number: 10 (2022).
Goodspeed et al., 2020. Brain Communications, Volume 2, Issue 2, 2020, fcaa170.
Greenblatt et al. (1989). Pharmacokinetic and electroencephalographic study of intravenous diazepam, midazolam, and placebo. Clin. Pharmacol. Ther. 45, 356-365.
Hipp et al., 2021. Basmisanil, a highly selective GABAA-α5 negative allosteric modulator: preclinical pharmacology and demonstration of functional target engagement in man Scientific Reports volume 11, Article number: 7700 (2021)
Hogart, A., Wu, D., LaSalle, J. M., and Schanen, N.C. (2010). The comorbidity of autism with the genomic disorders of chromosome 15q11.2-q13. Neurobiol. Dis. 38, 181-191.
Hyvarinen A (1999): Fast ICA for noisy data using Gaussian moments. In: Proceedings of the 1999 IEEE International Symposium on Circuits and Systems. ISCAS'99. vol. 5. Piscataway, NJ: IEEE, 57-61.
Judson et al., 2016. Neuron Volume 90, Issue 1, 6 Apr. 2016, Pages 56-69.
Jung et al. (2000): Removal of eye activity artifacts from visual event-related potentials in normal and clinical subjects. Clin Neurophysiol 111:1745-1758.
Kalsner, L., and Chamberlain, S. J. (2015). Prader-Willi, Angelman, and 15q11-q13 Duplication Syndromes. Pediatr. Clin. North Am. 62, 587-606.Keute et al. (2020). Angelman syndrome genotypes manifest varying degrees of clinical severity and developmental impairment. Molecular Psychiatry 1-9.
Knust et al. (2009) Bioorg Med Chem Lett. 2009 Oct. 15; 19(20):5940-4.
Liu et al., Bioorganic & Medicinal Chemistry. Volume 18, Issue 23, 1 Dec. 2010, Pages 8374-8382.
Lusk, L., Vogel-Farley, V., DiStefano, C., and Jeste, S. (1993). Maternal 15q Duplication Syndrome. In GeneReviews®, M. P. Adam, H. H. Ardinger, R. A. Pagon, S. E. Wallace, L. J. Bean, K. W. Gripp, G. M. Mirzaa, and A. Amemiya, eds. (Seattle (WA): University of Washington, Seattle), p.
Malizia, A. L. et al. Benzodiazepine site pharmacokinetic/pharmacodynamic quantification in man: Direct measurement of drug occupancy and effects on the human brain in vivo. Neuropharmacology 35, 1483-1491. https://doi.org/10.1016/s0028-3908(96) 00072-x (1996).
Mullen, E. M. (1995). Mullen Scales of Early Learning (AGS ed.). Circle Pines, MN: American Guidance Service Inc.
Prevot et al., 2019. Mol Neuropsychiatry 2019; 5:84-97.
Robertson, Ratai and Kanwisher, 2015. Curr Biol. 2016 Jan. 11; 26(1):80-5.
Rudolph, U., and Knoflach, F. (2011). Beyond classical benzodiazepines: novel therapeutic potential of GABAA receptor subtypes. Nat Rev Drug Discov 10, 685-697.
Sanders, et al. (2015). Insights into Autism Spectrum Disorder Genomic Architecture and Biology from 71 Risk Loci. Neuron 87, 1215-1233.
Santrac et al., 2022. Autism Research. 2022 May; 15(5): 806-820.
Saravanapandian et al (2020). Properties of beta oscillations in Dup15q syndrome. Journal of Neurodevelopmental Disorders 12, 22.
Savić, et al. “Are GABAA receptors containing α5 subunits contributing to the sedative properties of benzodiazepine site agonists?.” Neuropsychopharmacology 33.2 (2008): 332-339.
Schomer and Silva, 2012. Niedermeyer's Electroencephalography: Basic Principles, Clinical Applications, and Related Fields (7^{th} Ed.) Lippincott Williams & Wilkins, 2012.
Simeone et al., 2021. A Benzodiazepine Ligand with Improved GABA_{A} Receptor α5-Subunit Selectivity Driven by Interactions with Loop C. Molecular Pharmacology January 2021, 99 (1) 39-48.
Skolmick et al., J Pharmacol Exp Ther. 1997 November; 283(2):488-93.
Sparrow, S. S., Cicchetti, D. V., & Balla, D. A. (2005). Vineland adaptive behavior scales (2^{nd} ed.). Circle Pines, MN: American Guidance Service.
Sparrow, S. S., Cicchetti, D. V., & Saulnier, C. A. (2016). Vineland adaptive behavior scales, Third edition (Vineland-3). Bloomington: NCS Pearson.
Sternfeld et al., J Med Chem. 2004 Apr. 22; 47(9):2176-9.
Telias 2019. Front. Mol. Neurosci., 7 Mar. 2019. Sec.Neuroplasticity and Development.
Thibert et al. (2013): Neurologic manifestations of Angelman syndrome. Pediatr Neurol 48:271-279.
Trillingsgaard A, Østergaard JR (2004): Autism in Angelman syndrome: An exploration of comorbidity. Autism 8:163-174.
Urracaet al. (2013). The Interstitial Duplication 15q11.2-q13 Syndrome Includes Autism, Mild Facial Anomalies and a Characteristic EEG Signature. Autism Res. 6, 268-279. van Amerongen et al., (2019) Analgesic Potential of PF-06372865, an a2/a3/a5 Subtype-Selective GABAA Partial Agonist, in Humans. Br. J. Anaesth. 2019, 123 (2), 194-203. van Niel et al. (2005). A new pyridazine series of GABAA α5 ligands. J. Med. Chem. 2005; 48:6004-6011.
Visser, S. A., Wolters, F. L., van der Graaf, P. H., Peletier, L. A. & Danhof, M. Dose-dependent EEG effects of zolpidem provide evidence for GABAA receptor subtype selectivity in vivo. J. Pharmacol. Exp. Ther. 304, 1251-1257. (2003).
Violante et al., 2016. Neurology. Aug. 30, 2016; 87 (9).
Wang et al., 2018. Translational Psychiatry volume 8, Article number: 13 (2018).
Williams C A (1995): Angelman syndrome: Consensus for diagnostic criteria. Am J Med Genet 56:237-238.
Zureck et al., 2016. Ann Clin Transl Neurol. 2016 May; 3(5): 392-398.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only, and are not to be construed as limiting the disclosure in any way.

Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The terms “about” or “approximately” in relation to a numerical value is optional and means for example +/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

1. A computer-implemented method of determining whether a subject has a neurological dysfunction associated with a signal in a particular range of frequency of brain electrophysiological activity, the method comprising:
obtaining an EEG and/or MEG power spectrum from the subject; and
obtaining a metric quantifying the magnitude of the power in the power spectrum in the particular frequency range, wherein the metric is a value summarising the power spectrum in said frequency range corrected using an estimate of the power that is attributable to background signal, wherein the estimate of the power that is attributable to background signal is specific to said frequency range, and
wherein the metric is indicative of the presence and/or severity and/or directionality of a neurological dysfunction.

2. The method of claim 1, wherein the estimate of the power that is attributable to background signal comprises a plurality of anchor points wherein anchor points are points of the power spectrum located at or within a predetermined distance of the boundaries of the frequency range, or a curve in said frequency range of the power spectrum, optionally wherein the curve is linear, piecewise linear or non-linear and/or wherein the curve is derived from the plurality of anchor points or from a global fit of the power spectrum comprising a superposition of oscillatory signals and an aperiodic component.

3. The method of any preceding claim, wherein obtaining a metric quantifying the power in the power spectrum in the particular frequency range comprises:
determining a summarised estimate of the power in said frequency range that is attributable to background signal that is specific to said frequency range,
summarising the power in said frequency range, and
correcting the summarised power in said frequency range using the summarised estimate of the power in said frequency range that is attributable to background signal,
optionally wherein determining a summarised estimate of the power in said frequency range that is attributable to background signal comprises integrating the estimate of the power in said frequency range that is attributable to background signal over said frequency range and summarising the power in said frequency range comprises integrating the power over said frequency range, or
wherein summarising the power in said frequency range comprises selecting a minimum or maximum of the power in said frequency range or determining an average or weighted average of the power at one or more predetermined frequencies within said frequency range, and determining a summarised estimate of the power in said frequency range that is attributable to background signal comprises: (i) selecting the estimate of the power that is attributable to background signal at the frequency of the selected minimum or maximum of the power in said frequency range, or (ii) selecting a plurality of anchor points that are on the boundaries or outside of the frequency range and determining an average or weighted average of the signal at said anchor points, or
wherein summarising the power in said frequency range comprises selecting the value of the power at a predetermined frequency in said frequency range, and determining a summarised estimate of the power in said frequency range that is attributable to background signal comprises selecting the estimate of the power that is attributable to background signal at the predetermined frequency, optionally wherein the predetermined frequency has been previously determined using a plurality of reference power spectra, for example as the frequency at which the power is most likely to have a maximum or minimum value in said plurality of reference power spectra, or
wherein summarising the power in said frequency range and determining a summarised estimate of the power in said frequency range that is attributable to background signal comprise selecting the value of the power and the estimate of the power that is attributable to background signal at a frequency such that the corrected summarised power signal has the minimum or maximum value over the frequency range.

4. The method of claim 3, wherein correcting the summarised power in said frequency range using the summarised estimate of power in said frequency range attributable to the background signal in comprises subtracting the summarised estimate of power in said frequency range attributable to background signal from the summarised metric of the power signal in said frequency range or dividing the summarised power in said frequency range by the summarised estimate of power in said frequency range attributable to background signal.

5. The method of any preceding claim, wherein the estimate of the power in said frequency range that is attributable background signal is provided by interpolation between the power at two or more anchor points that are on the boundaries or outside of the frequency range, optionally linear interpolation, or wherein the summarised estimate of the power in said frequency range that is attributable to background signal is determined as a weighted average of the power signal at two or more points that are on the boundaries or outside of the frequency range,
and/or wherein:
one or more of said anchor points are outside of the frequency range,
one or more of said anchor points are selected in predetermined frequency ranges comprising a preceding frequency range and a subsequent frequency range around the frequency range, or
one or more of said anchor points are local optima (minima or maxima) in predetermined frequency ranges comprising a preceding frequency range and a subsequent frequency range around the frequency range,
optionally wherein when one or more of said anchor points are outside of the frequency range, they are:
within 4 or within 8 Hz of the nearest boundary of the frequency range,
within a distance to the nearest boundary of the frequency range that is ≤50%, ≤20%, ≤10% of the size of the frequency range, or
within 0.25 octaves or within 0.5 octaves of the nearest boundary of the frequency range,
within a distance to the nearest boundary of the frequency range that is ≤50%, ≤20%, ≤10% of the size of the frequency range as measured in octaves, or
located such that the range between the plurality of said anchor points includes at most a single complete frequency band and zero, one or two portions of a neighbouring frequency band,
optionally wherein the frequency range is about 16 to about 32 Hz and the anchor points are about 12 and about 40 Hz, and/or
optionally wherein the frequency range is about 16 to about 32 Hz and the anchor points are selected in a preceding frequency range that is about 12 to about 16 Hz and/or a subsequent frequency range that is about 32 to about 40 Hz.

6. The method of any of claims 1 to 4, wherein the estimate of the background signal in said frequency range is provided by the value of the 1/f^{α} component of a global fit of the power spectrum comprising a superposition of oscillatory signals and a 1/f^{α} component, in said frequency range.

7. The method of any preceding claim, wherein the EEG or MEG power spectrum is an EEG power spectrum, and/or wherein the power spectrum from the subject is an average or weighted average power spectrum over a plurality of sensors, and/or wherein obtaining a power spectrum from the subject comprises receiving a power spectrum or a plurality of power spectra from the subject from a user interface, memory, database, computing device or EEG/MEG data acquisition means, and/or wherein obtaining a power spectrum from the subject comprises receiving a plurality of power spectra comprising a power spectrum for each of a plurality of sensors and the method further comprises obtaining an average or weighted average of the plurality of power spectra.

8. The method of any preceding claim, wherein the power spectrum has been obtained by performing one or more of: obtaining one or more EEG/MEG recordings from the subject, pre-processing the one or more EEG/MEG recordings from the subject, and deriving one or more power spectra from said optionally pre-processed recordings, and/or
wherein the power spectrum is a pre-processed power spectrum, and/or
wherein the power spectrum is a log-scaled power spectrum, and/or
wherein the method comprises applying a non-linear transformation, and/or
wherein said frequency range:
is the range between 16 and 32 Hz,
comprises at least a portion of the beta-band,
primarily comprises the beta band,
comprises a portion of the beta-band and optionally one or more portion of one or more neighbouring bands wherein any neighbouring bands represents a minority of the said frequency range,
comprises frequencies in one frequency band,
comprises frequencies in a plurality of frequency bands, at most one of which is completely included in said frequency range,
optionally a log transformation, to the power spectrum prior to obtaining a metric quantifying the power signal in the power spectrum in the particular frequency range, optionally wherein pre-processing an EEG/MEG recording comprises one or more of: removing artefactual sections of the recording, wherein artefactual sections refer to signal in the EEG/MEG recording that is not related to brain activity, re-referencing of electrodes, filtering of the EEG/MEG recording, interpolating missing sections, and independent component analysis.

9. The method of any preceding claim, further comprising comparing the value of the metric with a control value or set of control values,
optionally wherein:
(i) the control value or set of control values correspond to the value(s) of the metric quantified from power spectrum/spectra obtained from healthy individuals, from typically developing individuals, or from subjects with a known neurological dysfunction, optionally wherein the value of the metric and/or the control value or set of values are adjusted based on the age of the subjects or subjects from which the control values were obtained, and/or wherein the subjects with a known neurological dysfunction comprise individuals with a known GABA-A dysfunction, optionally individuals with Dup15q syndrome, Angelman Syndrome, and/or a deletion of the PWAS critical region; or
(ii) the control value corresponds to the summarised estimate of the power in said frequency range that is attributable to background signal, and/or wherein the control value is the value at which the summarised power in said frequency range is equal to the summarised estimate of the power in said frequency range that is attributable to background signal, and/or
optionally wherein the comparison between the value of the metric and the control value or set of control values is indicative of whether the subject has a neurological dysfunction,
and/or is indicative of the extent and/or direction of the neurological dysfunction.

10. The method of any preceding claim, wherein the subject is a paediatric subject, wherein the subject is an adult subject, wherein the subject is a human subject, wherein the subject is a model animal, wherein the subject is a mammalian, wherein the subject is a neuronal cellular culture or brain organoid, wherein the subject is a subject who has been diagnosed as having or being at risk of having a neuropsychiatric disorder, wherein the subject is a subject who has been diagnosed as having or being at risk of having a neurodevelopmental disorder, wherein the subject is a subject who has been diagnosed as having or being at risk of having an autism spectrum disorder, wherein the power spectrum has been recorded over at least 2, 5 or 10 minutes of awake time, wherein the power spectrum is an EEG power spectrum recorded using at least 19 electrodes mounted according to the 10/20 system, wherein the power spectrum is an EEG power spectrum recorded using frontocentral electrodes, and/or wherein the power spectrum has been obtained while the subject is in resting state, and/or wherein the power spectrum has been obtained while the subject is awake, and/or wherein the filter of the EEG amplifier and/or the sampling rate of the EEG has been adjusted to capture electrophysiological signals in the frequency range.

11. A method of determining whether a subject has a GABA receptor dysfunction, optionally a GABA-A receptor dysfunction, the method comprising performing the method of any preceding claim using a power spectrum, preferably an EEG power spectrum, obtained from said subject, wherein said frequency region comprises at least a portion of the beta band of frequency and/or wherein the beta-band of frequency comprises frequencies between 12 and 32 Hz, wherein the metric is indicative of the presence and/or severity and/or direction of a GABA receptor dysfunction optionally wherein said frequency region comprises or consists of a range between about 16 Hz and about 32 Hz, and/or wherein a value of the metric that is higher or lower than a control value or set of control values, optionally wherein a control value is the value expected for a healthy or typically developing subject, indicates that the subject has excessive or deficient GABA receptor function, respectively, and/or wherein the magnitude of the difference between the value of the metric and a control value or set of control values indicates the severity of the dysfunction.

12. A method of determining whether a subject with a neuropsychiatric disorder is likely to benefit from treatment with a GABA-A activity modulating therapy, the method comprising performing the method of claim 11 using a power spectrum obtained from said subject, and determining whether the subject has a GABA-A receptor dysfunction, optionally an excessive or deficient GABA-A receptor function, wherein a subject that has a GABA-A receptor dysfunction is likely to benefit from treatment with a GABA-A activity modulating therapy, optionally wherein the subject is a subject who has been diagnosed as having an autism spectrum disorder or being likely to have an autism spectrum disorder.

13. The method of claim 12, wherein the GABA-A activity modulating therapy is a compound or composition, wherein the GABA-A activity modulating therapy is selective GABA-A activity modulating compound or composition, wherein the GABA-A activity modulating therapy is a GABA-A α5 receptor modulating therapy, wherein the GABA-A activity modulating therapy is a GABA-A positive modulator or negative modulator, and/or wherein the GABA-A activity modulating therapy is a GABA-A positive allosteric modulator or negative allosteric modulator, and/or wherein the method is performed prior to administering a GABA-A modulating therapy, and/or wherein the method is performed after administering a GABA-A modulating therapy, and/or wherein the method is performed after administering an acute GABA-A modulating therapy, and/or wherein the method is performed after administering a course of a GABA-A modulating therapy, and/or
wherein the method further comprises recommending a GABA-A activity modulating therapy or selecting the subject for treatment with a GABA-A modulating therapy or treating the subject with a GABA-A modulating therapy, optionally wherein a subject that is determined to have excessive GABA-A receptor function is recommended or selected for treatment or treated with a GABA-A negative modulator, and/or wherein a subject that is determined to have deficient GABA-A receptor function is recommended or selected for treatment or treated with a GABA-A positive modulator, and/or wherein a subject that is determined to not have deficient GABA-A receptor function is recommended or selected for treatment or treated with a therapy that is not a GABA-A modulating therapy, and/or
wherein the method is performed at least at two time points comprising a time point prior to administering a GABA-A modulating therapy and/or at one or more time points subsequent to administering a GABA-A modulating therapy, and the method comprises recommending that the GABA-A modulating therapy is discontinued or discontinuing a GABA-A modulating therapy if the comparison between the metric obtained at two time points does not indicate a reduction of GABA-A dysfunction after administering the GABA-A modulating therapy,
wherein the method is performed at least at two time points comprising a time point prior to administering a GABA-A modulating therapy and/or at one or more time points subsequent to administering a GABA-A modulating therapy, and the method comprises recommending that a GABA-A modulating therapy, such as but not limited to the GABA-A modulating therapy, is continued or continuing a GABA-A modulating therapy if the comparison between the metric obtained at two time points does not indicate a reduction of GABA-A dysfunction after administering the GABA-A modulating therapy,
wherein the method is performed using a power spectrum acquired after acute administration of a particular GABA-A activity modulating therapy and the method further comprises recommending a GABA-A activity modulating therapy or the particular GABA-A activity modulating therapy, recommending that the subject not be treated with a GABA-A activity modulating therapy or the particular GABA-A activity modulating therapy, selecting the subject for treatment without a GABA-A modulating therapy or the particular GABA-A activity modulating therapy, treating the subject with a course of treatment that does not comprise a GABA-A modulating therapy or the particular GABA-A activity modulating therapy, optionally wherein a subject that is determined to have GABA-A receptor dysfunction after acute administration of the GABA-A activity modulating therapy is recommended or selected for treatment or treated with a course of treatment that does not include a GABA-A activity modulating therapy, and/or wherein a subject that is determined not to have deficient GABA-A receptor function after acute administration of the GABA-A activity modulating therapy is recommended or selected for treatment or treated with a GABA-A activity modulating therapy.

14. A GABA-A modulating therapy for use in a method of treatment of a neuropsychiatric disorder in a subject, the method comprising:
(i) determining whether the subject has a GABA-A dysfunction or is likely to benefit from treatment with a GABA-A activity modulating therapy using the method of any of claims 11 to 13; and
(ii) administering the GABA-A modulating therapy to said subject if the subject is determined to have a GABA-A dysfunction or to be likely to benefit from treatment with a GABA-A activity modulating therapy,
optionally wherein the GABA-A modulating therapy is a positive modulator such as alogabat or a negative modulator such as basmisanil.

15. A system comprising:
a processor; and
a computer readable medium comprising instructions that, when executed by the processor, cause the processor to perform the steps of the method of any of claims 1 to 14,optionally wherein the system further comprise an EEG/MEG data acquisition means.
